(19) United States
(12) Patent Application Publication
(10) Pub. No.: US 2010/0081200 A1
(43) Pub. Date: Apr. 1, 2010

(54) FORMULATIONS AND METHODS FOR CULTURING STEM CELLS

(76) Inventors: Kristiina Rajala, Julkujarvi (FI); Marjo-Riitta Suuronen, Pirkkala (FI); Outi Hovatta, Espoo (FI); Heli Skottman, Siivikkala (FI)

Correspondence Address: SUGHRUE MION, PLLC, 2100 PENNSYLVANIA AVENUE, N.W., SUITE 800, WASHINGTON, DC, 20037, US

(21) Appl. No.: 12/632,057

(22) Filed: Dec. 7, 2009

Related U.S. Application Data
(63) Continuation of application No. PCT/FI2008/050331, filed on Jun. 4, 2008.
(60) Provisional application No. 60/924,908, filed on Jun. 5, 2007.

(30) Foreign Application Priority Data
Jun. 5, 2007 (FI) 20075417

(51) Int. Cl. C12N 5/02

(52) U.S. Cl. 435/377; 435/404; 435/378; 435/383

(57) ABSTRACT

The present invention relates to a serum replacement formulation and to a culture medium suitable for the derivation, maintenance and differentiation of stem cells.

### FIELD OF THE INVENTION

The present invention relates to xeno-free formulations for use in the derivation, maintenance and differentiation of stem cells, such as human embryonic stem cells.

### BACKGROUND OF THE INVENTION

Human embryonic stem cells (hESCs) are pluripotent cells that have the potential to differentiate into all cell types of a human body. Human ESCs are of great therapeutic interest because they are capable of indefinite proliferation in culture and are thus capable of supplying cells and tissues for replacement of failing or defective human tissue. There are high expectations that, in the future, human ESCs will be proliferated and directed to differentiate into specific cell types, which can be transplanted into human bodies for therapeutic purposes or used as cell models in drug discovery and toxicology studies.

Embryonic stem cells are difficult to maintain in culture because they tend to follow their natural cell fate and spontaneously differentiate. Most culture conditions result in some level of unwanted differentiation. Stem cells differentiate as a result of many intrinsic and extrinsic factors, including growth factors, extracellular matrix molecules and components, environmental stressors and direct cell-to-cell interactions. Long-term proliferative capacity, pluripotent developmental potential after prolonged culture and karyotypic stability are the key features with respect to the utility of stem cell cultures.

The undifferentiated stage of hESCs can be monitored by judging the morphological characteristics of the cells. Undifferentiated hESCs have a characteristic morphology with very small and compact cells. While some differentiated cells usually appear at the margin of colonies of hESCs, an optimal culture method provides growth support with minimal amount of differentiated cells. There are several biochemical markers that are used to track the status of undifferentiated stage of hESCs such as the transcription factor Oct4 and Nanog as well as cell surface markers TRA-1-60, TRA-1-81, SSEA-3/4. These markers are lost when hESCs begin to differentiate to any cell lineage.

Basic techniques to create and culture hESCs have been described. There are, however, limitations and drawbacks to many of the procedures currently used to culture hESCs. Embryonic stem cells have typically been derived and proliferated in culture medium containing animal serum (especially fetal bovine serum) or other animal derived products to permit the desired proliferation during such culturing. The presence of animal derived products in hESC culture media has several problems. Firstly, animal derived products may contain toxic proteins or immunogens that evoke an immune response in the recipient and thus lead to rejection upon transplantation (Martin et al., Nat Med. 2005 Feb.;11(2):228-32). Secondly, the use of animal products increases the risk of contamination by animal pathogens, such as viruses, mycoplasma and prions, which can pose a serious health risk in cell therapy and other clinical applications (Healy et al., Adv Drug Deliv Rev. 2005 Dec. 12;57(13):1981-8). In fact government agencies are increasingly regulating, discouraging and even forbidding the use of cell culture media containing animal derived products, which may contain such pathogens. Thirdly, undefined components in a cell culture compromise the repeatability of cell model experiments e.g. in drug discovery and toxicology studies.

To overcome the drawbacks of the use of serum or animal extracts, a number of serum-free media have been developed. Price et al. disclose in US Patent Publication 2002/0076747 a serum replacement, Knockout™ SR medium (Invitrogen, Carlsbad, Calif.), frequently used in hESC culture. This formulation, however, contains animal derived products, such as bovine serum albumin, and hence is not completely free of xeno-derived components. Several xeno-free serum replacements and media are currently available (X-Vivo 10, X-Vivo 20, SSS, Lipumin, Serex, Plasmanate, SR3). These serum replacements often are specifically formulated to support the culture of a single cell type. Furthermore, Thomson et al. disclose in US Patent Publication 2006/0084168 a serum- and xeno-free cell culture medium, which allegedly support the growth of ESCs in culture.

Unfortunately, Rajala et al. demonstrate in Hum. Reprod., 2007, 22(5):1231-1238, that all the above-mentioned formulations permit the cultivation of hESCs only for a few passages during an adaptation phase to a new medium without severe differentiation, followed by rapid differentiation upon subsequent passages.

Several feeder-free culture methods have been developed for hESCs. Many of these feeder-free methods utilize animal derived components. In addition, these methods suffer from inadequate reproducibility and currently are unable for long-term maintenance of undifferentiated hESCs with stable and normal normal karyotype. Feeder-free cultures with enzymatic passaging may also be so demanding for the hESCs that they become more prone to abnormalities.

Because of these problems associated with currently known culture media for hESCs, there is a great need for a defined xeno-free culture medium that reproducibly supports robust growth of hESCs for long-term without substantial differentiation while maintaining pluripotency and normal cell karyotype, and which is compatible with the expected regulatory guidelines governing clinical safety and efficacy as well as standardized methods for in vitro cell models used in drug discovery and toxicology validations.

BRIEF DESCRIPTION OF THE INVENTION

The present invention provides means and methods for derivation, maintenance and differentiation of clinical-grade stem cells. More specifically, the present invention relates to a serum replacement, a final culture medium comprising said serum replacements, and to methods for the uses thereof.

An object of the present invention is to provide a xeno-free serum replacement comprising at least one fatty acid selected from a group consisting of conjugated linoleic acid and eicosapentaenoic acid. In some embodiments, the concentration of conjugated linoleic acid (CLA) is such that a final culture medium, which is a basal medium supplemented with said serum replacement, comprises from about 0.5 mg/l to about 5 mg/l CLA, and the concentration of eicosapentaenoic acid (EPA) is such that the final culture medium comprises from about 1 mg/l to about 10 mg/l EPA.

In some embodiments, the serum replacement may further comprise Activin A and/or retinol. In some other embodiments, the concentration of Activin A is such that the final culture medium comprises from about 0.001 mg/l to about 0.02 mg/l Activin A and/or the concentration of retinol is such that the final culture medium comprises from about 0.25 mg/l to about 0.5 mg/l retinol.

In some still other embodiments, the serum replacement may further comprise stearic acid. In some further embodiments, the concentration of stearic acid is such that a final culture medium comprises from about 0.5 mg/l to about 5 mg/l stearic acid.

Another object of the present invention is to provide a xeno-free cell culture medium comprising a basal medium and a serum replacement according to the embodiments of the present invention.

A further object of the present invention is to provide a method for initiating a new stem cell line in vitro. Said method comprises a) providing isolated cells of desired origin, b) contacting said cells with the present xeno-free culture medium, and c) cultivating said cells under conditions suitable for stem cell culture. In some embodiments, the cultivation may be performed on a feeder cell layer. In some further embodiments, said isolated cells are of embryonic, adult somatic, or mesenchymal origin.

A still further object of the present invention is to provide a method for culturing stem cells. Said method comprises a) contacting said stem cells with the present xeno-free medium, and c) cultivating said cells under conditions suitable for stem cell culture. In some embodiments, the cultivation may be performed on a feeder cell layer. The present culture medium is able to support the maintenance and proliferation of stem cells in a substantially undifferentiated state over numerous in vitro passages. Additionally, the stem cells cultured in the culture medium according to the present invention are substantially undifferentiated, retain their pluripotency or multipotency and maintain their genomic integrity.

Furthermore, an object of the present invention is to provide a method for differentiating stem cells. Said method comprises a) contacting said stem cells with the present xeno-free medium supplemented with a differentiating agent, such as a growth factor or differentiating cells (e.g. END2 cells), and c) cultivating said cells under conditions suitable for differentiation of stem cells.

Other objects, embodiments, details and advantages of the present invention will become apparent from the following drawings, detailed description and examples.

### BRIEF DESCRIPTION OF THE DRAWINGS

In the following the invention will be described in greater detail by means of preferred embodiments with reference to the attached drawings, in which

FIGS. 1A and 1B are light microscopic images of hESC lines during a long-term culture in the present culture medium. FIG. 1A is a image of HS346 cells, passage 12; FIG. 1B is a image of HS401 cells, passage 10.

FIGS. 2A-2T show light and fluorescent microscopic images of hESCs cultured in different xeno-free culture media or serum replacements unable to maintain undifferentiated growth of the cells. One representative hESC colony after 1 passage in 20% Lipumin (FIG. 2A), in 20% Plasmanate (FIG. 2C), in 40% Plasmanate (FIG. 2E), in 20% SerEx (FIG. 2G), in 20% SR3 (FIG. 2I), in 20% SSS (FIG. 2K), in X-Vivo 10 (FIG. 2M), in X-Vivo 20 (FIG. 2O), and after 7 passages in TeSR1 (FIG. 2Q) or in control hES medium (FIG. 2S) are shown. The expression of Nanog and SSEA-1 in the corresponding hESC colonies are shown in FIGS. 2B, 2D, 2F, 2H, 2J, 2L, 2N, 2P, 2R, and 2T, respectively.

FIGS. 3A-3H illustrate hESCs during the adaptation phase in the present culture medium or in HEScGRO medium. FIG. 3A represents the adaptation phase 20:80 to HEScGRO medium. FIG. 3B represents the adaptation phase 50:50 to HEScGRO medium. FIG. 3C represents the adaptation phase 80:20 to HEScGRO medium. FIG. 3D represents hESCs after the adaptation phase to HEScGRO medium at passage 1. FIG. 3E represents the adaptation phase 20:80 to the present culture medium. FIG. 3F represents the adaptation phase 50:50 to the present culture medium. FIG. 3G represents the adaptation phase 80:20 to the present culture. FIG. 3H represents hESCs after the adaptation phase to the present culture at passage 1.

FIGS. 4A-4F show immunohistochemical stainings of hESC lines after long term culture in the present culture medium. FIG. 4A shows a staining of HS346 cells, passage 10, with Dapi. FIG. 4B shows a Nanog staining of HS346 cells, passage 10. FIG. 4C shows a SSEA3 staining of HS346 cells, passage 10. FIG. 4D shows a staining of HS401 cells, passage 7, with Dapi. FIG. 4E shows a Nanog staining of HS401 cells, passage 7. FIG. 4F shows a SSEA3 staining of HS401 cells, passage 7.

FIGS. 5A and 5B are light microscopic images of new hESC lines 06/015 (passage 6) and 07/046 (passage 51) respectively, after derivation and culture using the present culture medium.

FIGS. 6A, 6B, 6C and 6D show immunohistochemical stainings of new hESC lines 06/015 (passage 6) and 07/046 (passage 44) after derivation and culture using the present culture medium. FIG. 6A represents a Nanog staining of 06/015 cells, passage 6, FIG. 6B represents TRA-1-60 staining of 06/015 cells, passage 6, FIG. 6C represents a Nanog staining of 07/046 cells, passage 44, while FIG. 6D represents TRA-1-60 staining of 07/046 cells, passage 44.

FIG. 7 is a light microscopic image of a hESC culture in a standard hES medium with increased osmolarity.

FIGS. 8A-8D are light microscopic images of hESC line HS401 cultured in the present culture medium in different osmolarities for 5 passages: 260 mOsm (FIG. 8A), 290 mOsm (FIG. 8B), 320 mOsm (FIG. 8C) and 350 mOsm (FIG. 8D). Scale bar 500 μm.

FIGS. 9A-9B show the morphology and differentiation stage of hESCs cultured in the present culture medium and in the presence of lipids and lipid derivatives. UD, PD and DIFF represent undifferentiated, partly differentiated and differentiated hESC colonies, respectively. FIG. 9A) HS401 cell line cultured in the present culture medium supplemented with different lipids and lipid derivatives. FIG. 9B) HS401 cell line cultured in control hES medium supplemented with different lipids and lipid derivatives.

FIGS. 10A-10D demonstrate the increase in the proliferation and expression of stem cell markers in response to retinol. FIG. 10A) Bright-field microscopic image of hESCs (Regea 07/046) at day 3 cultured in the present culture medium without retinol for 5 passages. FIG. 10B) Bright-field microscopic image of hESCs (Regea 07/046) at day 3 cultured in the present culture medium containing 2.0 μM retinol for 5 passages. The size of the colonies is larger in the presence of retinol when compared to the colonies cultured without retinol. Scale bar 500 μm. FIGS. 10C-D) Fluorescent microscopic image of hESCs (HS401) cultured in the present culture medium containing 2.0 μM retinol for 12 passages showing positive expression of

Nanog and TRA-1-81. Insets represent DAPI staining. Scale bar 200 μm. FIG. 10E) Cell proliferation analysis of hESC line Regea 07/046 cultured in the present culture medium without and in the presence of 0.5, 2.0 and 3.5 μM retinol for 10 passages. FIG. 10F) Quantitative RT-PCR analysis of Oct4, GDF3, DNMT3B, TDGF1 and Nanog expression in hESC line Regea 07/046 cultured in the present culture medium without and in the presence of 0.5, 2.0 and 3.5 μM retinol for 10 passages.

FIGS. 11A-11C demonstrate the increase in the proliferation and expression of stem cell markers in response to Activin A. FIG. 11A) Cell proliferation analysis of hESC line Regea 07/046 cultured in the present culture medium without and in the presence of 5 ng/ml and 10 ng/ml Activin A and hES control medium for 10 passages. FIG. 11B) Quantitative RT-PCR analysis of Nanog, Oct4, GDF3, DNMT3B, GABRB3 and GDF3 expression in hESC line Regea 07/046 cultured in the present culture medium without and in the presence of 5 ng/ml and 10 ng/ml Activin A and hES control medium for 10 passages. FIG. 11C) FACS analysis of SSEA4 and TRA-1-60 stem cell markers of hESC line Regea 07/046 cultured in the present culture medium without and in the presence of 5 ng/ml and 10 ng/ml Activin A and hES control medium for 10 passages.

FIGS. 12A-12J show characterization of hESC lines derived and cultured for long-term in the present culture medium. FIG. 12A) A Giemsa band karyogram showing normal karyotypes of hESC lines, Regea 07/046 at passage 36, Regea 08/013 at passage 25, and Regea 06/040 at passage 71. FIG. 12B) Quantitative FACS analyses indicating expression of SSEA-4 and TRA-1-81 of hESC lines at day 7. Regea 07/046 at passage 45, Regea 08/013 at passage 41, and Regea 06/040 at passage 26. FIG. 12C) Cell proliferation analysis of hESC lines Regea 06/040 at passage 29, Regea 07/046 at passage 53 and Regea 08/013 at passage 41. FIG. 12D) Quantitative RT-PCR analysis of Nanog, Oct4, GABRB3, GDF3, DNMT3B and TDGF1 expression in hESC lines Regea 07/046 at passage 52, Regea 08/013 at passage 45, and Regea 06/040 at passage 33. FIG. 12E) Bright-field (scale bar, 500 μm) microscopic image showing undifferentiated colony morphology of hESC line 07/046 (p 33) 1 after freezing and subsequent thawing in the present culture medium at passage 1. FIG. 12F) RT-PCR analysis of in vitro-derived EBs showing transcripts for AFP and SOX-17 (endodermal markers), α-cardiac actin and T (Brachyury; mesodermal markers), SOX-1 and PAX6 (ectodermal markers), and β-actin as a housekeeping control. Lane 1, 50-bp DNA ladder. Regea 07/046 at passage 42, Regea 08/013 at passage 35, and Regea 06/040 at passage 101. FIG. 12G) Differentiated cardiomyocytes from hESC line Regea 08/013 stain positively with cardiac troponin T. Scale bar is 100 μm. FIG. 12H) Differentiated cardiomyocytes from hESC line Regea 08/013 stain positively with ventricular myosin heavy chain. Scale bar is 100 μm. FIG. 12I) RT-PCR analysis of neurospheres derived from hESC line Regea 08/013 cultured in the present culture medium showed expression of neural precursor markers Musashi, Nestin and PAX6; neuronal markers MAP-2, NF68 and OTX2; and astrocytic marker GFAP. No expression of pluripotent markers Oct4 and Nanog, nor endo- AFP or mesodermal markers T/Brachyury were detected. FIG. 12J) Most of the cells migrating out from the plated neurospheres stained positive for neuronal marker MAP-2 and few cells were positive for astrocytic marker GFAP. Scale bar is 100 μm.

FIGS. 13A-13C show characterization of human induced pluripotent stem cells (iPS cells) cultured in the present culture medium. FIG. 13A) Quantitative FACS analyses indicating expression of SSEA-4 and TRA-1-81 of human iPS cell lines cultured in hES medium and in the present culture medium. Cell samples cultured in hES medium are from 6 day old colonies, cell samples from iPS cell line A cultured in the present culture medium from 7 day old colonies and samples from iPS cell line B from 8 day old colonies. Cell line A in hES medium at passage 15, in the present culture medium at passage 14 and iPS cell line B in hES medium at passage 16, in the present culture medium at passage 7. FIG. 13B) Quantitative RT-PCR analysis of Nanog, Oct4, GABRB3, GDF3, DNMT3B and TDGF1 expression of day 6 colonies in iPS cell line A in hES medium at passage 10, in the present culture medium at passage 7 and iPS cell line B in hES medium at passage 11, in the present culture medium at passage 8. FIG. 13C) RT-PCR analysis of in vitro-derived EBs showing transcripts for AFP and SOX-17 (endodermal markers), α-cardiac actin and T (Brachyury; mesodermal markers), SOX-1 and PAX6 (ectodermal markers), and β-actin as a housekeeping control. Lane 1, 50-bp DNA ladder. Both cell lines at passage 10.

FIGS. 14A-14E show characterization of human adipose stem cells (ASCs) cultured in the present culture medium. FIG. 14A) Morphology of ASCs cultured in human serum (HS) containing medium at day 8. (Scale bar 500 μm). FIG. 14B) Morphology of ASCs cultured in the present culture medium at day 8. (Scale bar 500 μm). FIG. 14C) Morphology of ASCs cultured in HS medium at day 11. (Scale bar 500 μm). FIG. 14D) Morphology of ASCs cultured in the present culture medium at day 11. (Scale bar 500 μm). FIG. 14E) WST-1 proliferation assay. Proliferation of ASCs was examined in HS medium and in the present culture medium, and analyzed at time points 1, 4, 7, and 11 days. The data in diagram is presented as mean±SD. *p<0.05 (n=7 donors with 4 replicate wells).

### DETAILED DESCRIPTION OF THE INVENTION

Some embodiments of the present invention relate to a serum replacement formulation and to a culture medium comprising said serum replacement. Furthermore, some embodiments of the present invention relate to methods for stem cell derivation, culture, maintenance, and differentiation. Specifically, some embodiments of the invention provides a culture medium for stem cells, such as human embryonic stem cells (hESCs). Notably, said culture medium supports the maintenance and proliferation of stem cells, such as hESCs, in a substantially undifferentiated state. Advantageously, said culture medium supports maintenance and proliferation of stem cells, such as hESCs, over numerous in vitro passages. Additionally, the stem cells cultured in the present culture medium are substantially undifferentiated, retain their pluripotency and maintain their genomic integrity. For therapeutic applications, the present culture medium comprises no components, such as feeder cells, conditioned medium, serum or other medium components, purified from a non-human animal source. More preferably, the culture medium comprises components that are synthesized using recombinant or chemical methods.

Herein the term stem cells include both pluripotent and multipotent stem cells. Embryonic stem cells (ESCs) are pluripotent cells being able to differentiate into a wide variety of different cell types. Means and method for obtaining embryonic stem cells are available in the art. Blastomere biopsy is an attractive new technology which allows isolation and propagation of embryonic stem cells without damaging the donor embryo.

Induced pluripotent stem cells (iPS cells) are another example of pluripotent stem cells. iPS cells are generated from differentiated cells, typically from adult somatic cells such as fibroblasts by developmental reprogramming. Such cells have been described e.g. in WO 2008/151058 and US 2008/076176. iPS cells may be obtained by different methods available in the art.

Multipotent stem cells include, but are not limited to, hematopoietic stem cells and mesenchymal stem cells (MSCs), which are adult stem cells capable of differentiating into a variety of cell types. MSCs may be isolated from different sources including bone marrow and adipose tissue. MSCs derived from adipose tissue are termed as adipose stem cells (ASCs). Means and method for obtaining MSCs are available in the art.

The means and methods provided herein are applicable to stem cells derived from any desired animal, preferably mammals including primates such as humans, monkeys, and apes, as well as non-primate mammals such as such as mice, rats, horses, sheep, pandas, goats and zebras.

Some embodiments of the present invention provide a defined xeno-free serum replacement composition that may be used to supplement any suitable basal medium for use in the in vitro maintenance and proliferation of stem cells, preferably embryonic stem cells, such as primate (e.g. human) embryonic stem cells. Said serum replacement may be used to supplement both serum-free and serum-containing basal mediums, or any combinations thereof.

The serum replacement according to some embodiments of the present invention is suitable for maintaining and proliferating stem cells in a substantially undifferentiated state, while maintaining both the pluripotency and and the karyotype of the cells, for at least about 20 passages. In other embodiments, the maintenance of stem cells is supported for at least about 30, and preferably at least about 50 passages.

By the term xeno-free it is meant herein that the origin of the reagent is not from a foreign source, i.e. does not contain material of non-human animal origin when human stem cells are to be cultured. Likewise, culturing of, for instance, murine stem cells has to be done in the absence of any mice derived material in order to be xeno-free. Suitable xeno-free sources for culturing human stem cells may include chemical synthesis or synthetic preparations or isolation, preparation or purification of the reagent of interest from bacteria, yeasts, fungi, plants and humans.

By the term serum replacement it is meant herein a composition that may be used to replace animal serum in a final cell culture medium. A conventional serum replacement comprises typically vitamins, albumin, lipids, amino acids, transferrin, antioxidants, insulin and trace elements. The final cell culture medium may further comprise growth factors, non-essential amino acids, β-mercaptoethanol, L-glutamine and/or antibiotics added directly to the basal medium or further comprised in the serum replacement.

It has now been surprisingly found, that retinol (i.e. vitamin A) plays a crucial role in maintaining stem cells in an undifferentiated state. The effect of different vitamins on the undifferentiated growth of human embryonic stem cells was tested by providing retinol (20 μM), nicotinamide (5 mM and 10 mM) or commercial Vitamin Mix (1%) containing nicotinamide but not retinol (MEM Vitamin Solution (100x), cat. No. 11120-037, provided by Gibco/Invitrogen) to human embryonic stem cells (Table 1). Retinol increased the number of undifferentiated colonies considerably. Nicotinamide had the opposite effect to the embryonic stem cells, promoting their differentiation. Vitamin Mix had no effect on the undifferentiated growth of embryonic stem cells. Undesired results, i.e. differentiation of stem cells, have been previously reported with retinoic acid, a derivative of retinol, e.g. by Schuldiner et al. in Brain Res., 2001, 913(2):201-205, incorporated herein by reference.

TABLE 1
The effect of different vitamins on the undifferentiated
growth of human embryonic stem cells

| | | | Nicotin- | Nicotin- | MEM |
|---|---|---|---|---|---|
| Cell | | Vitamin A | amide | amide | Vitamin |
| line | Control | 20 μM | 10 mM | 5 mM | Mix 1% |
| HS346 | 0 | ++ | −− | − | 0 |
| 06/015 | 0 | +++ | −− | − | 0 |

In further experiments, it was found that even ten times lower concentrations of retinol, i.e. 2 μM, is effective in maintaining stem cells in an undifferentiated state. Accordingly, the serum replacement according to some embodiments of the present invention is a xeno-free formulation comprising at least retinol. In some embodiments, the concentration range of retinol in the serum replacement is such that the final culture medium comprises from about 0.25 mg/l to about 0.5 mg/l, more specifically about 0.57 mg/l retinol. Accordingly, in embodiments wherein basal medium is to be supplemented with 20% (vol/vol) serum replacement, said serum replacement comprises retinol from about 1.25 mg/l to about 2.5 mg/l, more specifically about 2.85 mg/l.

The serum replacement may further contain other vitamins such as ascorbic acid, biotin, choline chloride, D-Ca Pantothenate, Folic acid, i-inositol, niacinamide, Pyridoxal, Pyridoxine, Riboflavin, thiamine, Vitamin B 12, Vitamin D2. Typically several vitamins are included in the basal medium and additional vitamin supplementation can be added to the final medium. Suitable concentrations of vitamins in the serum replacement and the final medium according to some embodiments of the present invention, can be readily determined by a skilled person using routine methods well known in the art. Typically, thiamine is used in a concentration of about 9 mg/l, while ascorbic acid is used in a concentration of about 50 μg/ml in the cell culture medium according to some embodiments of the present invention.

Especially good results were obtained when retinol was used in combination with conjugated linoleic acid (CLA) and/or eicosapentaenoic acid (EPA), and even better results are obtained in the presence of Activin A.

Furthermore, it has now been surprisingly found that conjugated linoleic acid (CLA) and/or eicosapentaenoic acid (EPA) provide excellent results in maintaining stem cells in an undifferentiated state. Among various lipids and lipid derivates tested, these two fatty acids were superior in maintaining the undifferentiated morphology, increasing the number of undifferentiated colonies, and retaining the pluripotency or multipotency of stem cells.

Accordingly, the serum replacement according to some embodiments of the present invention is a xeno-free formulation comprising at least one fatty acid selected from the group consisting of conjugated linoleic acid and eicosapentaenoic acid. In some embodiments, the concentration range of CLA in the serum replacement is such that the final culture medium comprises from about 0.5 mg/l to about 5 mg/l, more specifically about 2.5 mg/l CLA. In some other embodiments, the concentration range of EPA in the serum replacement is such that the final culture medium comprises from about 1 mg/l to about 10 mg/l, more specifically about 5 mg/l EPA. Accordingly, in embodiments wherein a basal medium is to be supplemented with 20% (vol/vol) serum replacement in order to arrive at a final culture medium, said serum replacement comprises CLA from about 2.5 mg/l to about 25 mg/l, more specifically about 12.5 mg/l and/or EPA from about 5 mg/l to about 50 mg/l, more specifically 25 mg/l. It is evident to a person skilled in the art that the serum replacement may be provided in a form to be added to the basal medium with different percentages, whereby the concentrations of individual ingredients change accordingly.

The next best fatty acid for use in the present serum replacement is stearic acid. In some embodiments, the concentration range of stearic acid in the serum replacement is such that the final culture medium comprises from about 0.5 mg/l to about 5 mg/l, more specifically about 2.5 mg/l stearic acid. Accordingly, in embodiments wherein a basal medium is to be supplemented with 20% (vol/vol) serum replacement, said serum replacement comprises stearic acid from about 2.5 mg/l to about 25 mg/l, more specifically about 12.5 mg/l.

It has also been surprisingly found that Activin A, especially in combination with CLA and/or EPA promotes stem cell proliferation and expression of stem cell markers such as Nanog, Oct4, GDF3, DNMT3B, GABRB3 and GDF3.

Accordingly, the serum replacement according to some embodiments of the present invention may further comprise Activin A. In some embodiments, the concentration range of Activin A in the serum replacement is such that the final culture medium comprises from about 0.001 mg/l to about 0.02 mg/l, more specifically about 0.005 mg/l Activin A. Thus, in embodiments wherein a basal medium is to be supplemented with 20% (vol/vol) serum replacement, said serum replacement comprises Activin A from about 0.005 mg/l to about 0.1 mg/l, more specifically about 0.025 mg/l.

In some embodiments of the present invention, the serum replacement comprises Activin A and CLA and/or EPA. In some other embodiment, the serum replacement comprises retinol and CLA and/or EPA. In further embodiments, the serum replacement comprises Activin A, retinol, and CLA and/or EPA. Suitable concentrations of these ingredients are given above. Each and every serum replacement according to these embodiments may further comprise stearic acid.

In still further embodiments, the serum replacement may comprise in addition to the ingredients given above at least one ingredient, preferably free of endotoxins, selected from the group consisting of lipids or lipid derivatives, vitamins, albumins or albumin substitutes, amino acids, vitamins, transferrins, transferrin substitutes, antioxidants, insulin or insulin substitutes, trace elements, and growth factors. Such ingredients are to be present in the serum replacement formulation in a concentration sufficient to support the proliferation of stems cells in a substantially undifferentiated state, while maintaining both the pluripotency and the karyotype of the cells.

It has also been surprisingly found that fetuin and α-fetoprotein may be used to promote growth of stem cells. Table 2 shows the effect of fetuin and α-fetoprotein on growth rate and size of embryonic stem cell colonies. All formulations shown contained human serum albumin at a concentration of 10 mg/ml. Fetuin was shown to increase the colony size and growth rate the most at a concentration of 0.1 mg/ml and α-fetoprotein at a concentration of 0.05 mg/ml. When fetuin and α-fetoprotein were both included in the formulation, the growth promoting effect was even slightly better than in the formulations including them individually.

TABLE 2
Effect of fetuin and α-fetoprotein on the growth
rate and size of the embryonic stem cell colonies
(F = Fetuin, A = α-fetoprotein)

| | HS346 | 06/015 |
|---|---|---|
| Control | 0 | 0 |
| F 0.05 mg/ml | ++ | ++ |
| F 0.10 mg/ml | +++ | ++ |
| F 0.20 mg/ml | + | + |
| A 0.05 mg/ml | +++ | +++ |
| A 0.10 mg/ml | ++ | + |
| A 0.20 mg/ml | + | + |
| A 0.05 mg/ml + | +++ | +++ |
| F 0.10 mg/ml | | |

Accordingly, the serum replacement according to some embodiments of the present invention may further comprise fetuin, α-fetoprotein and/or any combination thereof. Fetuin and α-fetoprotein are commercially available fetal carrier proteins present at a high plasma concentration in fetal plasma. Fetuin and α-fetoprotein could be used to replace albumin in the serum replacement, but due to their high price it may be feasible to use them in combination with albumin. In some embodiments, the serum replacement comprises about 0.5 mg/ml fetuin and about 0.25 mg/ml α-fetoprotein. In such embodiments, a basal medium is to be supplemented with 20% serum replacement. In general, a typical final cell culture medium comprises from about 0.01 mg/ml to about 1 mg/ml fetuin and/or α-fetoprotein.

Albumin substitutes suitable for use in the present serum replacement include any compound, which may be used instead of albumin and has essentially similar effects as albumin. Suitable concentration of albumin or albumin substitute in the serum replacement and in the final culture medium according to some embodiments of the present invention, can be readily determined by a skilled person using routine methods well known in the art. Typically, albumins or albumin substitutes are used in the final medium in the range of about 1 mg/ml to about 20 mg/ml, preferably of about 5 mg/ml to about 15 mg/ml. In one embodiment, albumin is present at about 10 mg/ml in the cell culture medium according to the present invention.

The serum replacement according to some embodiments of the present invention may further comprise at least one lipid or lipid derivative including but not limited to lipoproteins such as very-low-density lipoprotein (VLDL), low-density lipoprotein (LDL), high-density lipoprotein (HDL) and cholesterol; phospholipids such as phosphatidylcholine, lysophosphatidylcholine, phosphatidylserine, phosphatidylinositol, sphingomyelin, and phosphatidylethanolamine; fatty acids such as linoleic acid, gamma-linoleic acid, linolenic acid, arachidonic acid, oleic acid, docosahexaenoic acid, palmitic acid, palmitoleic acid, myristic acid and their derivatives such as prostaglandins. According to various embodiments of the present invention, the serum replacement may comprise e.g. at least two, at least three or at least four of the lipids or lipid derivatives given above.

A person skilled in the art can readily determine suitable concentrations of lipids and lipid derivatives for use in the present serum replacement using standard methods known in the art.

Amino acids suitable for use in the present serum replacement include, but are not limited to amino acids, such as glycine, L-histidine, L-isoleucine, L-methionine, L-phenylalanine, L-proline, L-hydroxyproline, L-serine, L-threonine, L-tryptophan, L-tyrosine, L-valine, and their D-forms and derivatives. Suitable concentrations of amino acids can be readily determined by a skilled person using routine methods well known in the art. Typical concentration ranges are presented in Table 3. The serum replacement according to some embodiments of the present invention may contain additional non-essential amino acids, such as L-alanine, L-asparagine, L-aspartic acid, L-glutamic acid, glycine, L-proline, L-serine, and their D-forms and derivatives. Such additional non-essential amino acids may be included in the present serum replacement or added directly to the present final cell culture medium. Non-essential amino acids may be provided as a commercially available mixture, such as MEM non-essential amino acids (NEAA) provided by Invitrogen. Typically, the concentration of said mixture in the final culture medium is about 1%.

L-glutamine is preferably added to the cell culture medium according to the present invention as a stabilized, dipeptide form of L-glutamine such as Glutamax (Invitrogen, 2 mM). When desired, L-glutamine may be included in the serum replacement according to some embodiments of the present invention.

Transferrins are involved in iron delivery to cells, controlling free iron concentration in biological fluids and preventing iron-mediated free radical toxicity. Suitable transferrin substitutes for use in the present serum replacement include any compound which may be used instead of transferrin and has essentially similar effects as transferrin. Such substitutes include, but are not limited to, iron salts and chelates (e.g., ferric citrate chelate or ferrous sulfate). Suitable concentrations of transferrin or transferrin substitute in the serum replacement and the final medium according to some embodiments of the present invention, can be readily determined by a skilled person using routine methods well known in the art. Typically, suitable range of transferrin or transferrin substitute in the final culture medium is about 1 μg/ml to about 1000 μg/ml, preferably about 5 μg/ml to about 100 μg/ml, and more preferably, about 5 μg/ml to about 10 μg/ml. In one embodiment, transferrin is present at about 8 μg/ml in the final cell culture medium.

Antioxidants suitable for use in the present serum replacement include, but are not limited to glutathione and ascorbic acid. Suitable concentrations of antioxidants in the serum replacement and the final medium according to some embodiments of the present invention can be readily determined by a skilled person using routine methods well known in the art. According to one embodiment, glutathione is present at 1,5 μg/ml and ascorbic acid is present at 50 μg/ml in the final cell culture medium.

Insulin substitutes suitable for use in the present serum replacement include any compound, which may be used instead of insulin and has essentially similar effects as insulin. Suitable concentration of insulin or insulin substitute in the serum replacement and the final medium according to some embodiments of the present invention can be readily determined by a skilled person using routine methods well known in the art. Typically, suitable range of insulin in the final medium is about 1 μg/ml to about 1000 μg/ml, preferably about 1 μg/ml to about 100 μg/ml, more preferably about 50 μg/ml to about 15 μg/ml. In some embodiments, insulin is present at about 10 μg/ml.

Trace elements suitable for use in the present serum replacement include, but are not limited to Mn²⁺, Si⁴⁺, Mo⁶⁺, V⁵⁺, Ni²⁺, Sn²⁺, Al³⁺, Ag⁺, Ba²⁺, Br⁻, Cd²⁺, Co²⁺, Cr³⁺, F⁻, Ge⁴⁺, I⁻, Rb⁺, Zr⁴⁺ and Se⁴⁺ and salts thereof. Suitable concentrations of trace elements or salts thereof can be readily determined by a skilled person using routine methods known in the art. Commercially available trace element compositions such as Trace Elements B and C provided by CellGro Mediatech Inc. may also be used. When desired, trace elements Cu²⁺ and/or Zn²⁺ may be included e.g. in the form of a commercially available Trace Element A composition provided by CellGro Mediatech Inc.

Furthermore, the present inventors have shown that lithium chloride may be harmful for embryonic stem cells resulting in differentiation thereof. Thus, in some specific embodiments, the serum replacement is devoid lithium chloride.

Growth factors suitable for use in the present serum replacement include fibroblast growth factors (FGFs) such as basic FGF (bFGF or FGF-2). Suitable range of FGF in final medium according to some embodiments of the present invention is about 1 ng/ml to about 1000 ng/ml, preferably about 2 ng/ml to about 100 ng/ml, and more preferably about 4 ng/ml to about 20 ng/ml. In one embodiment, FGF is present at about 8 ng/ml. While FGF is preferably used, other materials, such as certain synthetic small peptides (e.g. produced by recombinant DNA variants or mutants) designed to activate fibroblast growth factor receptors, may be used instead of FGF. Growth factors may be included in the present serum replacement or they may be added separately to the present final cell culture medium.

Antibiotics can also be used, to avoid contamination of the present serum replacement or the final culture medium. Suitable antibiotics or combinations thereof, as well as suitable concentrations are apparent to a person skilled in the art. However, if the medium is to be used in the culture of cells for clinical applications one might want to avoid the use of antibiotics.

Furthermore, β-mercaptoethanol may be included in the serum replacement according to some embodiments of the present invention or it may be added separately into the final culture medium according to some embodiments of the present invention. Typically, the final concentration of β-mercaptoethanol is about 0.1 mM in the culture medium.

In obvious embodiments of the present invention, any of the components of the serum replacement described above may be added directly into a basal medium to provide a final cell culture medium instead of being provided in the present serum replacement.

Some embodimenst of the present invention further provide a defined xeno-free culture medium for the in vitro maintenance and proliferation of stem cells, preferably embryonic stem cells. Said culture medium comprises a basal medium and a serum replacement composition set forth herein. Suitable basal media for use in the present culture medium include, but are not limited to KnockOut Dulbecco's Modified Eagle's Medium (KO-DMEM), Dulbecco's Modified Eagle's Medium (DMEM), Minimal Essential Medium (MEM), Basal Medium Eagle (BME), RPMI 1640, F-10, F-12, a Minimal Essential Medium (aMEM), Glasgow's Minimal Essential Medium (G-MEM), Iscove's Modified Dulbecco's Medium and HyQ ADCF-MAb (HyClone) and any combinations thereof. According to some preferred embodiments the basal medium is KO-DMEM. The term “basal medium” refers to any medium which is capable of supporting growth of stem cells, and in general supplies standard inorganic salts, vitamins, glucose, a buffer system and essential amino acids. In some embodiments, the basal medium may be supplemented with about 1 g/L to about 3.7 g/L sodium bicarbonate. In further embodiments, the basal medium is supplemented with about 2.2 g/L sodium bicarbonate.

The osmolarity of the culture affects to the success and vitality of stem cell cultures. Osmolarity, measured in milli-osmoles, is a measure of the number of dissolved particles in a solution, which is a measurement of the osmotic pressure that a solution will generate. Normal human serum has an osmolarity of about 290 milli-osmoles. Media for in vitro culture of other mammalian cells vary in osmolarity, but some media have an osmolarity as high as 330 milli-osmoles. Preferably, the osmolarity of the medium according to some embodiments of the present invention is between about 280 and about 330 mOsmol. However, osmolarity of the medium can be as low as about 260 mOsmol and as high as about 340 mOsmol. In some embodiments, hESCs are grown in an osmolarity of about 320-330 milli-osmoles.

According to some embodiments, lipids, albumin, amino acids, vitamins, transferrin, antioxidants, insulin, and trace elements are included in the serum replacement, while growth factors, non-essential amino acids, β-mercaptoethanol, L-glutamine and antibiotics are added directly to the cell culture medium. Final compositions according to some embodiments of the present culture medium is exemplified in Table 3.

TABLE 3
Final compositions of culture media according to some embodiments of the present invention

| | | | | | | Typical |
|---|---|---|---|---|---|---|
| | Concentration in some culture medium formulations (mg/l) | | | | | concentration |
| Ingredient | 1 | 2 | 3 | 4 | 5 | range (mg/ml) |
| Fatty acids* | | | | | | |
| Linoleic acid | 1 | | | | | 0-1000 |
| Arachidonic acid | 1 | | | | | 0-1000 |
| Oleic acid | 1 | | | | | 0-1000 |
| Sphingosine-1- | 10 μM | | | | | 0-20 μM |
| phosphate | | | | | | |
| Conjugated | | 2.5 | 2.5 | 2.5 | 2.5 | 0-1000 |
| linoleic acid and/or | | | | | | |
| Eicosapentaenoic | | 5 | 5 | 5 | 5 | 0-1000 |
| acid | | | | | | |
| Amino acids* | | | | | | |
| Glycine | 53 | 53 | 53 | 53 | 53 | 0-200 |
| L-histidine | 183 | 183 | 183 | 183 | 183 | 0-250 |
| L-isoleucine | 615 | 615 | 615 | 615 | 615 | 0-700 |
| L-methionine | 44 | 44 | 44 | 44 | 44 | 0-200 |
| L-phenylalanine | 336 | 336 | 336 | 336 | 336 | 0-400 |
| L-proline | 600 | 600 | 600 | 600 | 600 | 0-1000 |
| L-hydroxyproline | 15 | 15 | 15 | 15 | 15 | 0-100 |
| L-serine | 162 | 162 | 162 | 162 | 162 | 0-250 |
| L-threonine | 425 | 425 | 425 | 425 | 425 | 0-500 |
| L-tryptophan | 82 | 82 | 82 | 82 | 82 | 0-100 |
| L-tyrosine | 84 | 84 | 84 | 84 | 84 | 0-100 |
| L-valine | 454 | 454 | 454 | 454 | 454 | 0-500 |
| Vitamins* | | | | | | |
| Thiamine | 9 | 9 | 9 | 9 | 9 | 0-20 |
| Retinol | 20 μM | 0.57 μM | | 0.57 μM | | 0-100 μM |
| Antioxidants* | | | | | | |
| Glutathione | 1.5 | 1.5 | 1.5 | 1.5 | 1.5 | 0-20 |
| Ascorbic acid | 50 | 50 | 50 | 50 | 50 | 0-200 |
| Proteins | | | | | | |
| Human serum | 10000 | 10000 | 10000 | 10000 | 10000 | 0-50000 |
| albumin* | | | | | | |
| Fetuin* | 100 | | | | | 0-1000 |
| α-fetoprotein* | 50 | | | | | 0-1000 |
| Insulin* | 10 | 10 | 10 | 10 | 10 | 0-200 |
| Transferrin* | 8 | 8 | 8 | 8 | 8 | 0-200 |
| FGF | 0.008 | 0.008 | 0.008 | 0.008 | 0.008 | 0.004-0.5 |
| Activin A | | | | 0.005 | 0.005 | |
| Trace elements* | | | | | | |
| MnSO₄•H₂O | 0.17 | 0.17 | 0.17 | 0.17 | 0.17 | 0-10 |
| Na₂SiO₃•9H₂O | 140 | 140 | 140 | 140 | 140 | 0-200 |
| Molybdic acid | 1.24 | 1.24 | 1.24 | 1.24 | 1.24 | 0-10 |
| Ammonium salt | | | | | | |
| NH₄VO₃ | 0.65 | 0.65 | 0.65 | 0.65 | 0.65 | 0-10 |
| NiSO₄•6H₂O | 0.13 | 0.13 | 0.13 | 0.13 | 0.13 | 0-10 |
| SnCl₂ (anhydrous) | 0.12 | 0.12 | 0.12 | 0.12 | 0.12 | 0-10 |
| AlCl₃•6H₂O | 1.20 | 1.20 | 1.20 | 1.20 | 1.20 | 0-10 |
| AgNO₃ | 0.17 | 0.17 | 0.17 | 0.17 | 0.17 | 0-10 |
| Ba(C₂H₃O₂)₂ | 2.55 | 2.55 | 2.55 | 2.55 | 2.55 | 0-10 |
| KBr | 0.12 | 0.12 | 0.12 | 0.12 | 0.12 | 0-10 |
| CdCl₂ | 2.28 | 2.28 | 2.28 | 2.28 | 2.28 | 0-10 |
| CoCl₂•6H₂O | 2.38 | 2.38 | 2.38 | 2.38 | 2.38 | 0-10 |
| CrCl₃ (anhydrous) | 0.32 | 0.32 | 0.32 | 0.32 | 0.32 | 0-10 |
| NaF | 4.20 | 4.20 | 4.20 | 4.20 | 4.20 | 0-10 |
| GeO₂ | 0.53 | 0.53 | 0.53 | 0.53 | 0.53 | 0-10 |
| KI | 0.17 | 0.17 | 0.17 | 0.17 | 0.17 | 0-10 |
| RbCl | 1.21 | 1.21 | 1.21 | 1.21 | 1.21 | 0-10 |
| ZrOCl₂•8H₂O | 3.22 | 3.22 | 3.22 | 3.22 | 3.22 | 0-10 |
| Selenium | 0.00001 | 0.00001 | 0.00001 | 0.00001 | 0.00001 | 0.00000-0.1 |
| Other ingredients | | | | | | |
| NEAA | 1% | 1% | 1% | 1% | 1% | 0-10% |
| L-glutamine | 2 mM | 2 mM | 2 mM | 2 mM | 2 mM | 1-2 mM |
| β-mercaptoethanol | 0.1 mM | 0.1 mM | 0.1 mM | 0.1 mM | 0.1 mM | 0-1 mM |
| antibiotics | 50 U/ml | 50 U/ml | 50 U/ml | 50 U/ml | 50 U/ml | 0-100 U/ml |
| Basal medium | | | | | | |

Ingredients marked with an asterisk are provided in the form of a serum replacement according to some embodiments of the present invention.

TABLE 4
Effects of different ingredients on stem cells

| | Effect on | Effect on | Effect on |
|---|---|---|---|
| | stem cell | stem cell | stem cell |
| | morphology | proliferation | self-renewal |
| Serum replacement | +++ | +++ | ++ |
| including retinol | | | |
| Serum replacement | + | +++ | +++ |
| including Activin A | | | |
| Serum replacement | ++ | + | ++ |
| including CLA | | | |
| Serum replacement | ++ | + | +++ |
| including EPA | | | |
| Serum replacement | +++ | ++++ | ++++ |
| incl. retinol, Activin A | | | |
| Serum replacement | +++++ | ++++ | +++++ |
| incl. retinol, Activin A, | | | |
| CLA and/or EPA | | | |

Based on the results obtained, the best formulation for derivation and maintenance of stem cells is a formulation comprising retinol, Activin A and CLA and/or EPA, such as formulation 4.

The serum replacement or the culture medium according to some embodiments of the present invention may be provided in a liquid or a dry form. Furthermore, they may be provided as any suitable concentrated formulation. As an example, basal medium may be supplemented with 10%, 15% or 20% (vol/vol) serum replacement so as to result in final concentrations of ingredients as given above. When desired, ingredients of the serum replacement or the medium may be divided into compatible subformulations.

In some embodiments, the present invention provides a method for culturing and maintaining clinical-grade stem cells in a xeno-free culture. Said method comprises contacting stem cells with the culture medium according to some embodiments of the present invention, and cultivating said cells under conditions suitable for stem cell culture. Such conditions are apparent to a person skilled in the art. Stem cells may be maintained in an undifferentiated state over numerous in vitro passages in the present formulations. More specifically, said formulations may be used for maintaining and proliferating stem cells for at least about 20, preferably at least about 30, and more preferably at least about 50 passages. As demonstrated in Example 9, stem cells have been successfully maintained in a substantially undifferentiated state for even over 80 passages. Said stem cells retain their pluripotency, or multipotency. For instance, embryonic stem cells maintain their potential to differentiate into derivatives of endoderm, mesoderm and ectoderm tissues. Furthermore, said stem cells retain their genomic integrity as judged e.g. by their unchanged karyotypes.

For therapeutic applications, the culture medium according to some embodiments of the invention comprises no components, such as feeder cells, conditioned medium, serum or other medium components, purified from a non-human animal source. In some embodiments, the culture medium comprises components that are synthesized using recombinant or chemical methods.

The present compositions and methods are useful in the culturing of stem cells including embryonic stem cells such as primate embryonic stem cells. Preferably, primate embryonic stem cells that are cultured using this method are hESCs that are true embryonic stem cell lines in that they: (i) are capable of indefinite proliferation in vitro in an undifferentiated state; (ii) are capable of differentiation to derivatives of all three embryonic germ layers (endoderm, mesoderm, and ectoderm), even after prolonged culture; and (iii) maintain a normal karyotype throughout prolonged culture. Embryonic stem cells are, therefore, referred to as being pluripotent.

Stem cells that may be cultured in the medium according to some embodiments of the present invention may be from any animal, preferably mammals and more preferably, primates. Preferred cell types that may be cultured in a substantially undifferentiated state using the defined culture medium of the invention include stem cells derived from humans, monkeys, and apes. With regard to human stem cells, hESCs are preferred. hESCs may be derived from an embryo, preferably from a pre-implantation embryo, such as from a blastula or a morula. Stem cells derived from non-primate mammals, such as mice, rats, horses, sheep, pandas, goats and zebras, mayn also be cultured in the present culture medium. While in some embodiments the culture medium may be used for culturing embryonic stem cells, in other embodiments it may be used for culturing adult stem cells, such as, but not limited to, hematopoietic stem cells (HSGs) and adipose stem cells (ASCs). The art is replete with information of both embryonic and adult stem cells. Stem cells, including hESCs, cultured in accordance with the embodiments of present invention may be obtained from any suitable source using any appropriate technique, including, but not limited to, immunosurgery. For example, procedures for isolating and growing human embryonic stem cells are described in U.S. Pat. No. 6,090,622. Procedures for obtaining Rhesus monkey and other non-human primate embryonic stem cells are described in U.S. Pat. No. 5,843,78 and international patent publication WO 96/22362. In addition, methods for isolating Rhesus monkey embryonic stem cells are described by Thomson et al., (1995, Proc. Natl. Acad. Sci. USA, 92:7844-7848).

The present culture medium may also be used for studying, identifying and/or screening molecules, such as drug candidates, which i) affect the proliferation of undifferentiated stem cells, ii) affect the differentiation of stem cells, and iii) regulate tissue regeneration. The culture medium may also be used in a method for producing various agents, such as therapeutic proteins, in genetically modified stem cells or differentiated cells obtained therefrom.

The serum replacement and the final culture medium may further be used in a method of differentiating stem cells into a desired clinical-grade linage, especially for therapeutic purposes. This may be achieved by adding appropriate and sufficient differentiating agents into the present culture medium. Non-limiting examples of differentiating agents include Noggin, which may be used to differentiate oligodentrocytes; sonig hedgehog and retinoic acid, which may be used to differentiate motor neurons; bFGF, which may be used to differentiate retinal cell lineages; and BMP2, which may be used to differentiate cardiomyocytes; Activin A and IGF2, which may be to differentiate insulin-producing cells; and Activin A, BMP2 and BMP4, which may be used to differentiate hepatic cells. Furthermore, the differentiating agent may be a differentiating cell, such as END2, which may be used to differentiate cardiomyocytes. Other differentiating agents are well known in the art. Accordingly, this aspect of the present invention provides a method for differentiating stem cells into a desired linage. The method comprises contacting stem cells with the culture medium according to the present invention supplemented with a differentiating agent, and cultivating said cells under conditions suitable for stem cell culture. Current differentiation protocols utilize a variety of undefined products and culture media that may have unknown effects to the cell characteristics and differentiation. The present formulations, differentiation methods and uses do not share these disadvantages.

The present invention further provides a method for initiation, i.e. derivation or initiation, of new stem cell lines, such as ESCs, ASCs and iPS cell lines. The method comprises the steps of providing isolated cells of desired origin, contacting said cells with a xeno-free medium according to some embodiments of the present invention, and cultivating said cells under conditions suitable for cell culture. In some embodiments, the medium is supplemented with laminine, such as human placental laminine, and fibronectin, such as human plasma fibronectin. In further embodiments, laminine and fibronectin are used in a concentration of about 5 μg/ml.

The compositions and methods according to some embodiments of the present invention may optionally be used for culturing and/or initiating stem cell lines on a feeder cell layer. Suitable feeder cells include but are not limited to fibroblasts, such as human foreskin fibroblasts, e.g. CRL-2429 (ATCC, Mananas, USA).

In some embodiments, the present compositions and methods are used for feeder cell-free culture of stem cells.

### EXAMPLE 1

### Human ESCs Cultured in a Xeno-free Culture Media According to Some Embodiments of the Present Invention Remain Morphologically Undifferentiated

Three hESC lines HS237, HS346 and HS401 (Hovatta et al., Hum Reprod. 2003 Jul.;18(7):1404-9, Inzunza et al., Stem Cells. 2005 Apr.;23(4):544-9) were initially derived and cultured in a standard hES medium (disclosed in US 2002/0076747) containing 80% (vol/vol) KnockOut DMEM (Gibco Invitrogen, Carlsbad, Calif., USA) supplemented with 20% (vol/vol) KnockOut Serum Replacement (ko-SR, Invitrogen), 2 mM Glutamax (Invitrogen), 0.1 mM β-mercaptoethanol (Invitrogen), 0.1 mM MEM non-essential amino acids (Cambrex Bio Science), 50 U penicillin/ml-50 μg streptomycin/ml (Cambrex Bio Science) and 8 ng/ml recombinant human basic fibroblast growth factor (bFGF, R&D Systems, Minneapolis, Minn., USA). Commercially available human foreskin fibroblast cells (CRL-2429, ATCC, Mananas, USA) were used as feeder cells.

Human ESC were gradually adapted to test culture conditions using an increasing proportion of the culture medium according to some embodiments of the present invention (with ratios of said culture medium to the standard hES media at 20:80, 50:50, 80:20) up to 100% during four weeks of culture. The culture medium according to this embodiment of the present invention contained bFGF (8 ng/ml; R&D Systems), human serum albumin (10 mg/ml; Sigma or Vitrolife), insulin (10 ug/ml; Invitrogen), transferrin (8 ug/ml; Sigma), Glutathione (1.5 μg/ml, Sigma), Thiamine hydrochloride (9 μg/ml, Sigma), Ascorbic acid (50 μg/ml, Sigma), Amino acids (as listed in Table 3), Trace elements B and C (1:1000, Cellgro, Herndon, Va., USA), linoleic acid (1 μg/ml, Sigma), arachidonic acid (1 μg/ml, Cayman Chemicals), oleic acid (1 μg/ml, Cayman Chemicals), retinol (20 μM, Sigma), sphingosine-1 phosphate (10 μM, Sigma) in KODMEM, further supplemented with 2 mM Glutamax, 0.1 mM MEM non-essential amino acids and 0.1 mM β-mercaptoethanol. All medium components were synthetic, recombinant or of human origin. Osmolarity was adjusted to 320-330 mOsm/Kg with 5 M NaCl. Cells were mechanically passaged every 6-8 days to new mitotically inactivated feeder cells.

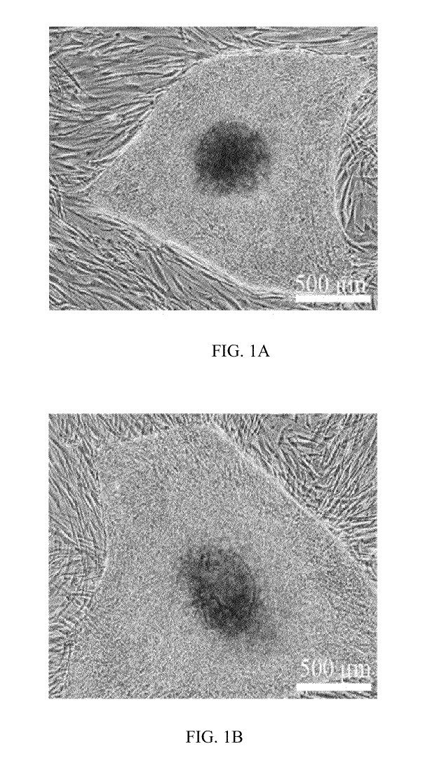

To determine whether hESCs grown in the present culture medium were maintained in an undifferentiated state, the morphology of the cells was examined after every passage. Human embryonic stem cell line HS237 was maintained in the present culture medium at least for 23 passages, HS346 for at least 15 passages and HS401 for at least 17 passages. The morphology of hESC lines remained undifferentiated after long-term culture in the present culture medium (FIG. 1).

Similar results were obtained with culture media according to other embodiments of the present invention.

### EXAMPLE 2

### Comparison of a Culture Medium According to Some Embodiments of the Present Invention to HesGro and Other Commercially Available Xeno-free Serum Replacements

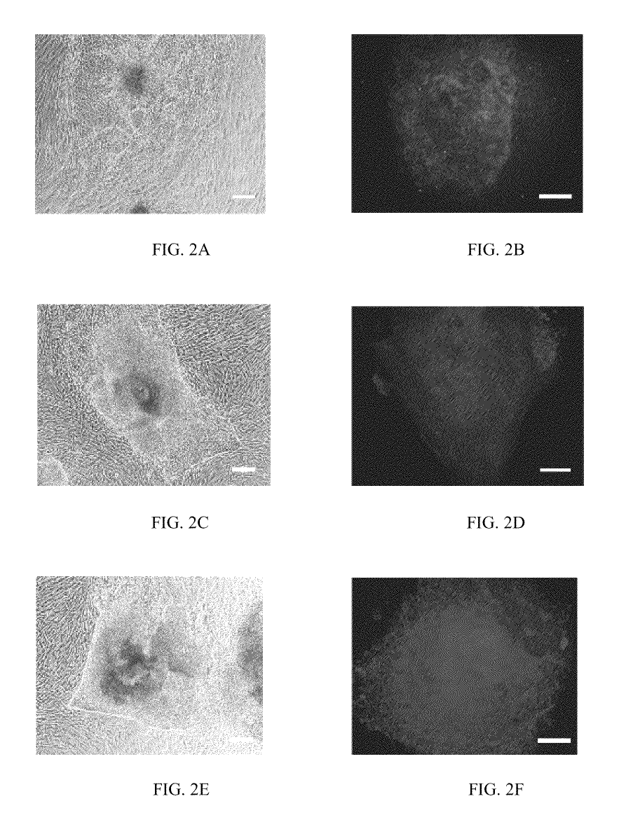

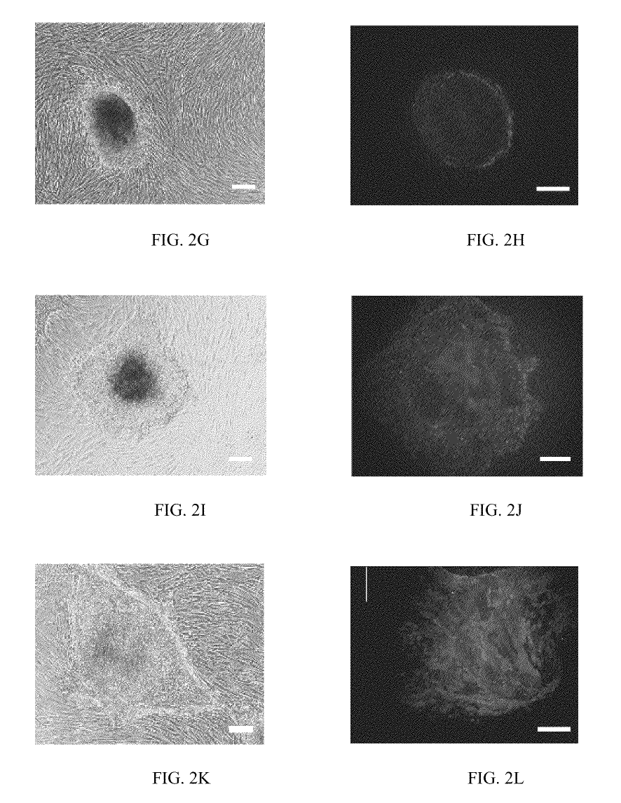

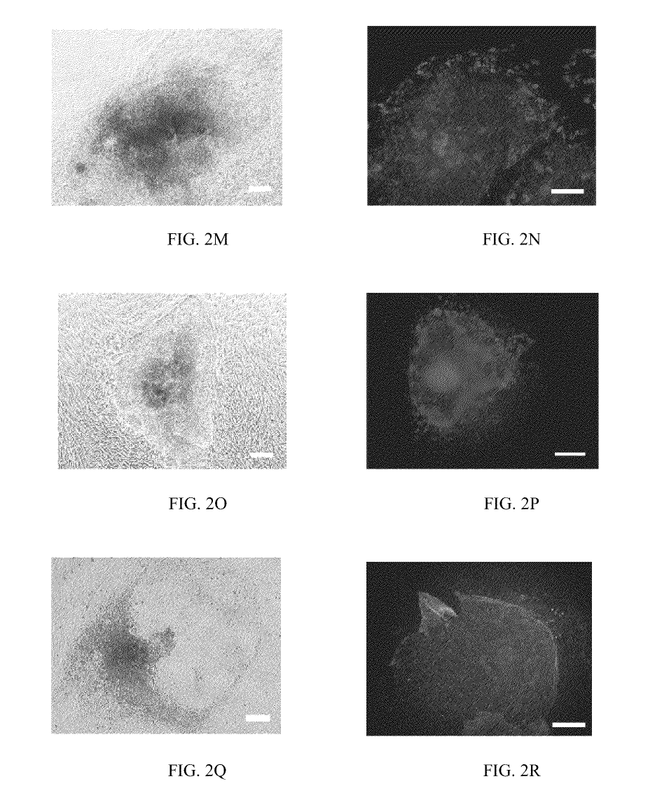

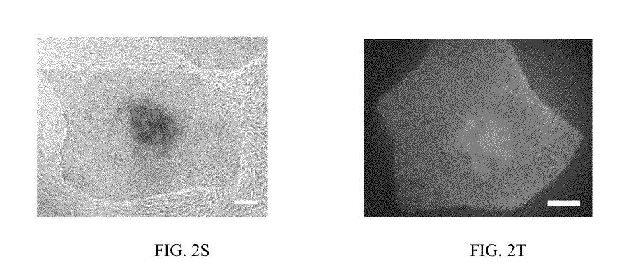

In order to test different culture conditions and the suitability of the culture conditions for long-term maintenance of human ESCs, an evaluation assay was performed in which hESCs were cultured under different xeno-free test conditions. The test conditions, cell lines and passage numbers employed are listed in Table 5. Human ESCs were gradually adapted to different test media using an increasing proportion of test media (with ratios of test media to hES media at 20:80, 50:50, 80:20) up to 100% test media during the four weeks of culture. The differentiation was first judged by morphology and then confirmed by immunofluoresence analysis. The hESC colonies grown in the commercially available culture media (Lipumin, SerEx, SSS, SR3, TeSR1, Plasmanate, X-vivo10, X-vivo 20 and human serum) showed an increased expression of a marker common to the differentiated hESC (SSEA-1, 1:200, Santa Cruz Biotechnology, Inc., Santa Cruz, Calif., USA) and were negative to a marker common to the undifferentiated hESCs (Nanog, 1:200, Santa Cruz Biotechnology) (FIG. 2). Human ESC line HS237 cultured in hES medium was used as a control in immunofluoresence analysis (FIG. 2).

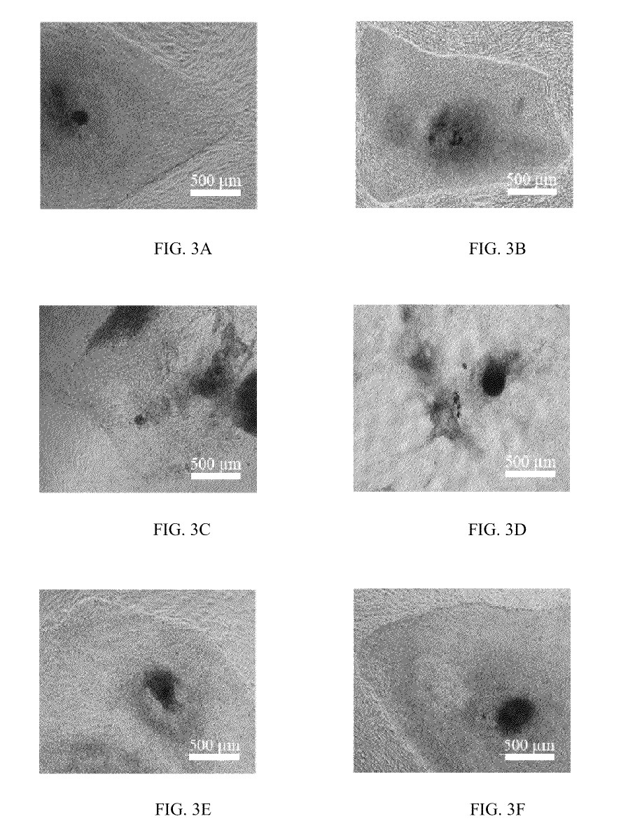

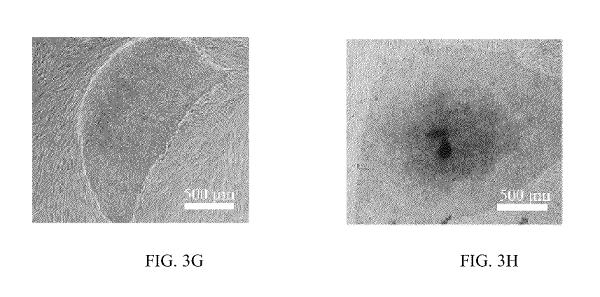

The culture medium according to one embodiment of the present invention (described in Example 1) was also compared to a xeno-free commercially available proprietary HEScGRO medium (Chemicon) developed for hESCs. HEScGRO medium was unable to maintain undifferentiated state of hESCs. The differentiation began already during the adaptation phase with hESCs cultured with HEScGRO medium (FIG. 3). The results clearly showed that HEScGRO medium is not able to maintain the undifferentiated growth of hESCs. Only the present culture medium of the xeno-free culture media tested was able to maintain the undifferentiated growth of hESCs on human feeder cells.

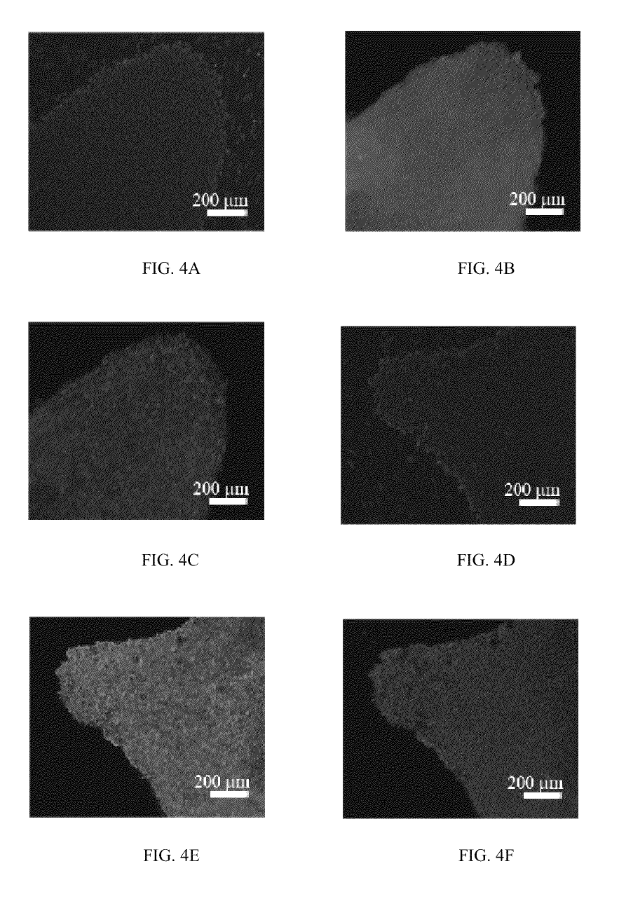

To confirm that hESCs grown in the present culture medium for long term remain undifferentiated, the expression of stem cell markers Nanog (1:200, Santa Cruz Biotechnology), SSEA3 (1:200, Santa Cruz Biotechnology) was examined (FIG. 4).

Similar results were obtained with culture media according to other embodiments of the present invention.

TABLE 5
The test conditions, cell lines and passage numbers employed

| | Cell line and | |
|---|---|---|
| Test reagent | starting passage | Medium composition^{a} |
| Control hES medium | HS181 p62 | 80% ko-DMEM; |
| | HS237 p59, p74 | 20% ko-SR |
| | HS293 p49 | |
| | HS306 p50 | |
| Lipumin ™ 10x | HS181 p62 | 80/90% ko-DMEM; |
| | HS237 p59, p74 | 10/20% Lipumin |
| | HS293 p49 | |
| Plasmanate | HS181 p62 | 80/60% ko-DMEM; |
| | HS237 p74 | 20/40% Plasmanate |
| SerEx 10x | HS181 p62 | 80/90% ko-DMEM; |
| | HS237 p59 | 10/20% SerEx |
| | HS293 p49 | |
| Serum Substitute | HS181 p62 | 80/90% ko-DMEM; |
| Supplement SSS | HS237 p59, p74 | 10/20% SSS |
| | HS293 p49 | |
| SR3 | HS181 p60 | 80/90% ko-DMEM; |
| | HS237 p61 | 10/20% SR3 |
| | HS293 p42 | |
| TeSR1 | HS237 p74 | DMEM/F12; |
| | HS181 p62 | 16.5 mg/ml HSA; |
| | | 108 μg/ml transferrin; |
| | | 196 μg/ml insulin; |
| | | 6 mg/L thiamine HCl; |
| | | 41.5 mg/L LiCl; |
| | | 2 mg/L glutathione; |
| | | 50 mg/L L-ascorbic acid; |
| | | 1:1000 trace elements B |
| | | and C solution; |
| | | 0.1 mg/ml GABA; |
| | | 0.02 mg/L sodium |
| | | selenite; |
| | | 0.127 μg/ml pipecolic |
| | | acid; |
| | | 0.6 ng/ml TGF-β1; |
| | | 1:500 chemically defined |
| | | lipid concentrate |
| X-Vivo 10 | HS237 p59 | 100% X-vivo10; |
| | HS293 p49 | 0.12 ng/ml TGFβ1 |
| X-Vivo 20 | HS181 p60 | 100% X-vivo20 |
| | HS237 p61 | |
| HEScGRO | HS346 p68-p71 | 100% HEScGRO |
| | HS401 p77-p80 | |
| Medium of the present | HS237 p80-p103 | As described in example |
| invention | HS346 p67-p81 | 1 |
| | HS401 p75-p91 | |

^{a}In all other cases except HEScGRO, the test medium is supplemented with 2 mM Glutamax, 0.1 mM β-mercaptoethanol, 0.1 mM MEM non-essential amino acids, 50 U penicillin/ml-50 μg streptomycin/ml, and 8 ng/ml bFGF. Appreviations: Ko-DMEM, KnockOut Dulbecco's modified Eagle medium; ko-SR, KnockOut Serum Replacement; DMEM/F12, Dulbecco's modified Eagle medium: F12 Nutrient mixture; HSA, human serum albumin; LiCl, litium chloride; GABA, γ-aminobutyric acid; TGF-β1, Transforming growth factor- β1.

### EXAMPLE 3

### Characterization of Pluripotency (RT-PCR) and Karyotyping during Long-term Culture of Several hESC Lines

To confirm that hESCs cultured in the present culture medium still maintain their pluripotency in vitro, embryoid body formation and differentiation assays of HS237, HS346 and HS401 cells were performed. Subsequently, the embryoid bodies (EBs) continued to differentiate on plates for at least 20 days. The EBs were formed by mechanically dissecting hESC colonies and transferring the resulted pieces onto a culture dish without feeder cells. The EBs were cultured in the present culture medium without bFGF for at least 20 days before the isolation of RNA. The hESC cultured in a standard hES medium were used as a control and samples were prepared similarly.

Total RNA was isolated from EBs using RNeasy mini kit (Qiagen, Valencia, Calif., USA). The RNA extraction was performed according to the manufacturer instructions. Complementary DNA (cDNA) was synthesized from 50 ng of total RNA using Sensiscript Reverse Transcription Kit (Qiagen) according to manufacturer instructions. The expression of markers characteristic of ectoderm (neurofilament 68 KD), endoderm (α-fetoprotein) and mesoderm (α-cardiac actin) development in EBs were determined using RT-PCR primers (Proligo, Sigma). Glyseraldehyde 3-phosphate dehydrogenase (GAPDH) was used as a housekeeping control. The negative control contained sterilized water instead of cDNA template. The PCR reactions were carried out in the Eppendorf Mastercycler as follows: denaturation at 95° C. for 3 minutes and 40 cycles of denaturation at 95° C. for 30 s, annealing at 57° C. for 30 s and extension at 72° C. for 1 minute, followed by final extension at 72° C. for 5 minutes. The PCR products were analyzed with electrophoresis on 1.5% agarose gel containing 0.4 μg/ml ethidium bromide (Sigma) and DNA standard (MassRuler™ DNA Ladder Mix, Fermentas). In all hESC lines the EBs contained cells from three different lineages (Table 6). Hence, the present culture medium was sufficient to maintain the pluripotency of hESCs.

TABLE 6
RT-PCR analysis of embryoid bodies differentiated from
HS237, HS346 and HS401 lines cultured in the culture medium
according to some embodiments of the present invention

| Embryonic | | | | |
|---|---|---|---|---|
| layer | Gene | HS237 | HS346 | HS401 |
| ectoderm | neurofilament 68KD | + | + | + |
| endoderm | α-fetoprotein | + | + | + |
| mesoderm | α-cardiac actin | + | + | + |
| | GAPDH | + | + | + |

No major translocations or other chromosomal changes were observed in karyotyping of the hESCs. Thus, hESCs cultured in the present culture medium maintain their genomic integrity.

### EXAMPLE 4

### Derivation of New hESC Lines using the Present Culture Medium

Using the present culture medium, we have been able to derive new hESC line from surplus bad quality human embryo donated for stem cell research. A prior and informed consent was obtained from the donors of the embryos used in the derivation of new embryonic stem cell lines. Furthermore, Regea, Institute for Regenerative Medicine, University of Tampere, Finland has the approval of the Ethical Committee of Pirkanmaa Hospital District to derive and culture hESC lines.

This media as described in example 1 highly supported the derivation of new hESC lines. In addition this medium enabled the derivation procedure without any immunosurgery methods e.g using mechanical isolation of cells from embryo. Moreover, this medium enabled the derivation procedure using human fibroblasts cultured without animal-derived media thus suitable for production of hESC for clinical applications under GMP-standards and without any trace of animal-derived components. At derivation procedure, the media can be supplemented with 5 μg/ml human placental laminine and human plasma fibronectin to increase attachment of cells during derivation process.

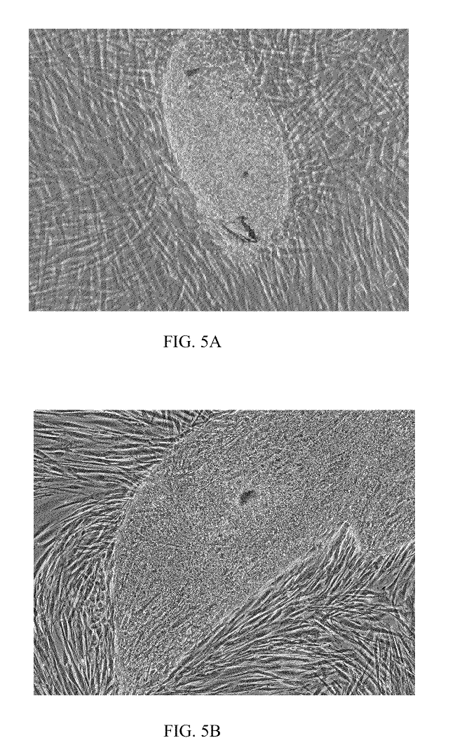

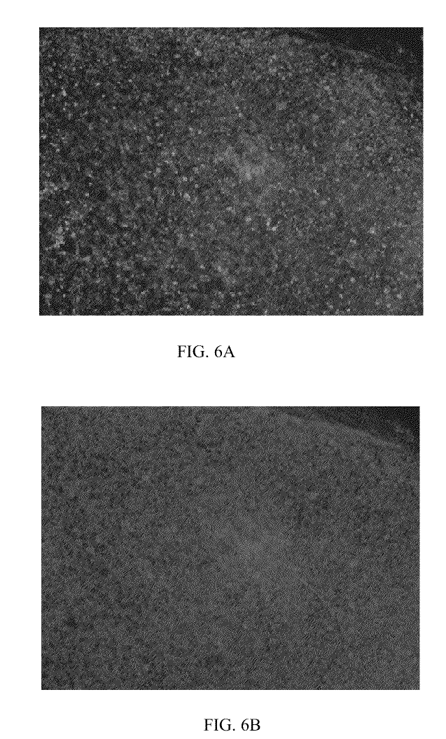

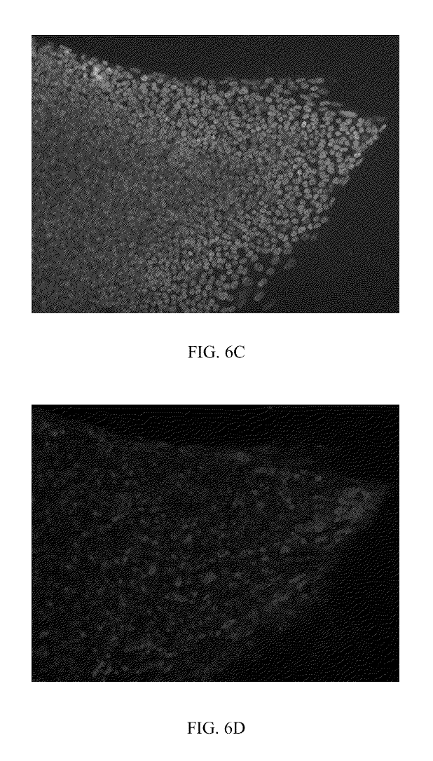

To determine whether new hESC lines were growing in the present culture medium and maintained undifferentiated state, the morphology of the cells was examined after every passage (FIG. 5). The new hESC lines derived using said culture medium were characterized by immunocytochemical staining with several markers specific for undifferentiated hESC (FIG. 6) and pluripotency of the lines was determined with in vitro embryoid body formation as described above. In addition, the derived new hESC lines were determined to have maintained normal karyotype for 06/015 cells at passage 16 and for 07/046 cells at passages 20 and 44.

The composition of the formulation according to this embodiment of the present invention was further optimized as described above in Table 2. It was found that fetuin and α-fetoprotein may be used to promote growth of stem cells. Fetuin was shown to increase the colony size and growth rate the most at a concentration of 0.1 mg/ml and α-fetoprotein at a concentration of 0.05 mg/ml. When fetuin and α-fetoprotein were both included in the formulation, the growth promoting effect was even slightly better than in the formulations including them individually.

### EXAMPLE 5

### Characterization of the Effect of Osmolarity on hESCs

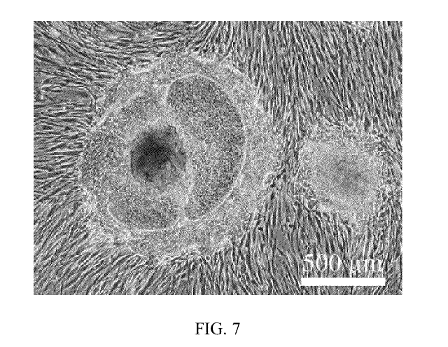

To further demonstrate that the osmolarity of the medium for culturing hESCs should be less than 350 mOsm/kg, hESCs were cultured and monitored in the standard hES medium. Osmolarity of the medium was raised to 350 mOsm/kg with 5 M NaCl. The proliferation of hESCs decreased rapidly and excessive differentiation was observed. hESCs were maintained in hES medium with osmolarity of 350 mOsm/kg for 4 passages. HESCs showed reduced proliferation and excessive differentiation after 3 passages (FIG. 7). On the other hand, hESCs cultured in an osmolarity of 326 mOsm/kg remained undifferentiated.

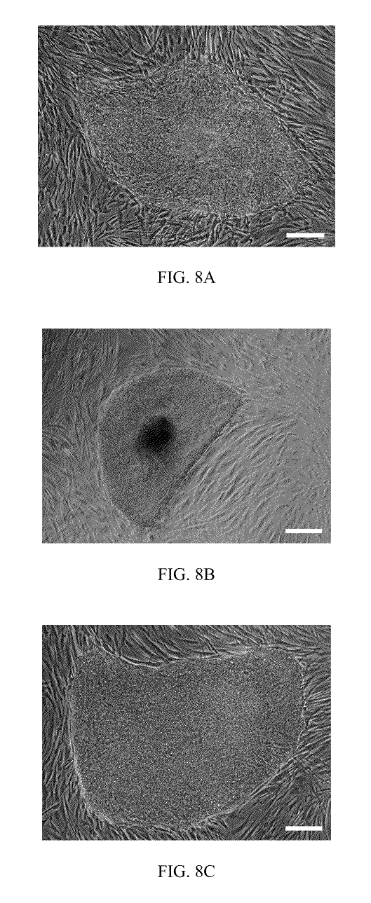

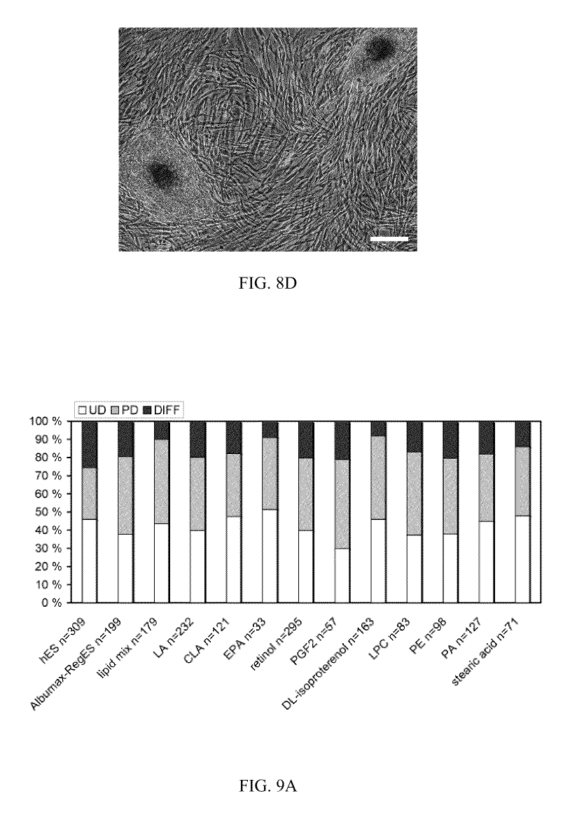

Furthermore, the osmolarity of the present culture medium comprising retinol and Activin A was adjusted with 5 M NaCl. Various different osmolarities were tested in the culture of human embryonic stem cells (hESCs) for 5 passages and the morphology of the cells was examined after every passage. The best performance was obtained with osmolarity of 320 mOsm. With the omolarity of 260 mOsm small uneven colonies were formed and even though the morphology of the colonies was improved with the osmolarity of 290 mOsm the size of the colonies was small. The osmolarity of 350 mOsm clearly restricted the growth of the colonies (FIG. 8).

### EXAMPLE 8

### Specific Lipids and Lipid Derivatives Enhance the Undifferentiated Growth of hESCs

Various lipids and lipid derivatives were tested in the culture medium (RegES) according to some embodiments of the present invention and in the conventional hES culture medium containing KO-SR (Knockout serum replacement, Invitrogen). General morphology, as well as the size and thickness of the undifferentiated colonies were evaluated before each passaging based on visual perceptions (Table 7). According to the results conjugated linoleic acid, eicosapentaenoic acid, palmitoleic acid, linoleic acid, linoleic-oleic-arachidonic acid mix and especially retinol improved the morphology of the undifferentiated colonies in both hES medium and in the present culture medium (Table 7). In addition, stearic acid, lysophosphatidylcholine, phosphatidylethanolamine, prostaglandin F₂ and DL-isoproterenol resulted in poor morphology and/or excess differentiation in hES culture medium whereas in the present culture medium these supplements resulted in satisfying morphology.

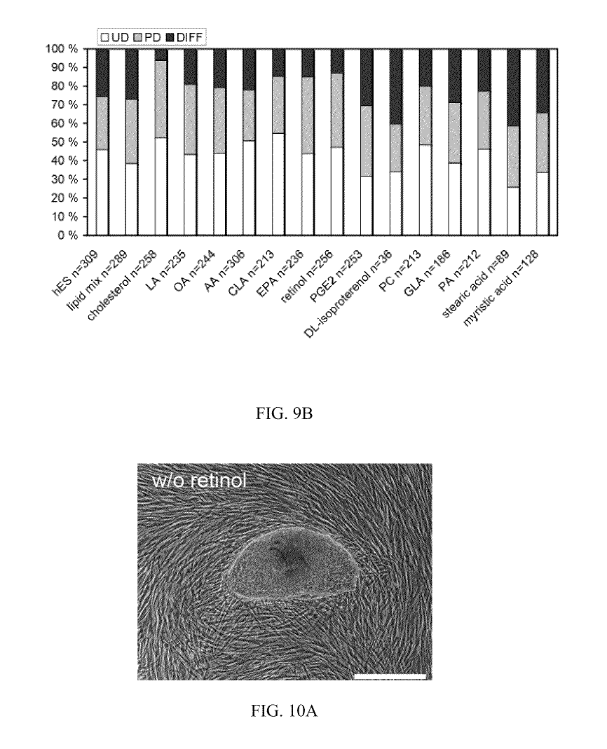

Furthermore, the hESC colonies were classified into three categories; undifferentiated, partly differentiated and differentiated. Number of each colony type was calculated before each passaging. Later, a percentage value for each colony type of the total amount of colonies was calculated (FIG. 9). In the present culture medium the number of undifferentiated colonies increased and the number of differentiated colonies decreased in the presence of conjugated linoleic acid, eicosapentaenoic acid, stearic acid, retinol, linoleic-oleic-arachidonic acid mix, DL-isoproterenol, palmitoleic acid and linoleic acid when compared to the colonies cultured in the control hES or Albumax-RegES medium containing Albumax (Invitrogen) instead of human serum albumin. In hES culture medium the number of undifferentiated colonies increased and the number of differentiated colonies decreased in the presence of cholesterol, arachidonic acid, conjugated linoleic acid, retinol and phosphatidylcholine when compared to the colonies cultured in the control hES medium.

It was found that retinol and conjugated linoleic acid overall improved the colony morphology and the number of undifferentiated colonies in both culture media. In addition to retinol and conjugated linoleic acid; eicosapentaenoic acid, resulted in excellent performance by increasing the number of undifferentiated colonies in the culture present culture medium. Thus, conjugated linoleic acid and eicosapentaenoic acid are the most preferred fatty acids to be included in the present serum replacement. The third best performance was observed with stearic acid in the culture medium according to some embodiments of the present invention.

TABLE 7
Evaluated lipids and lipid derivatives

| | | | | Morphology* |
|---|---|---|---|---|
| Group | Common name/Abbr | Conc | | hES/RegES |
| Saturated FAs | Myristic acid | 2.5 | μg/ml | +/− |
| | Stearic acid | 2.5 | μg/ml | +/++ |
| Unsaturated FAs | Palmitoleic acid, PA | 2.5 | μg/ml | ++/++ |
| | Oleic acid, OA | 2.5 | μg/ml | +++/− |
| | Linoleic acid, LA | 2.5 | μg/ml | ++/++ |
| | Conjugated linoleic acid, CLA | 5 | μg/ml | +++/++ |
| | Gamma-linoleic acid, GLA | 2.5 | μg/ml | ++/− |
| | Alfa-linoleic acid, ALA | 5 | μg/ml | −/− |
| | Arachidonic acid, AA | 2.5 | μg/ml | +++/− |
| | Eicosapentaenoic acid, EPA | 5 | μg/ml | ++/++ |
| | Docosahexaenoic acid, DHA | 5 | μg/ml | −/− |
| | Linoleic-oleic-arachidonic acid mix | 2.5 | μl/ml | ++/++ |
| Phospholipids | Phosphatidylcholine, PC | 2.5 | μg/ml | ++/− |
| | Lysophosphatidylcholine, LPC | 5 | μg/ml | −/++ |
| | Phosphatidylethanolamine PE | 5 | μg/ml | −/++ |
| Sphingolipid | Sphingosine-1-phosphate, S1P | 10 | μM | −/− |
| Eicosanoids | Prostaglandin E₂, PGE₂ | 50 | ng/ml | +/− |
| | Prostaglandin F₂, PGF₂ | 50 | ng/ml | −/++ |
| Sterol | Cholesterol | 2 | μg/ml | ++/− |
| Vitamin A | Retinol | 2.5 | μg/ml | +++/+++ |
| Catecholamine | DL-isoproterenol | 0.1 | mg/ml | +/++ |

*General morphology, size and thickness of the undifferentiated colonies were evaluated. − excess differentiation of the colonies, poor morphology. + poor morphology, uneven edges in the colonies, thin and/or small colonies. ++ satisfying morphology, some uneven edges may exist in the colonies, colonies have medium thickness and size. +++ nice morphology, even, thick and big colonies.

### EXAMPLE 7

### Retinol Increases Proliferation and Expression of Stem Cell Markers

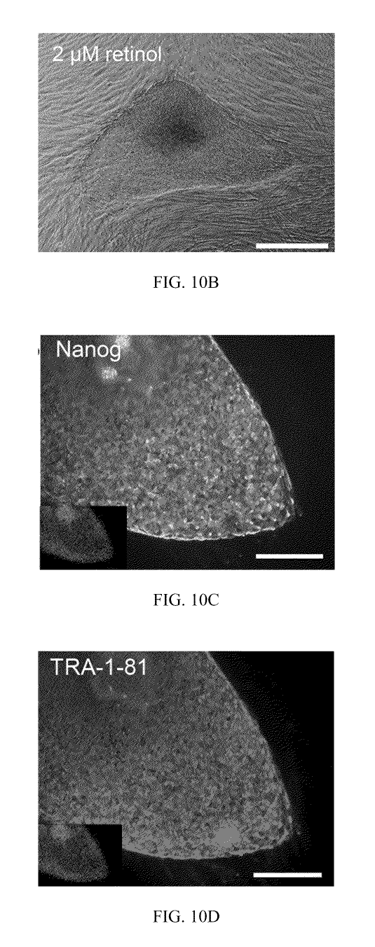

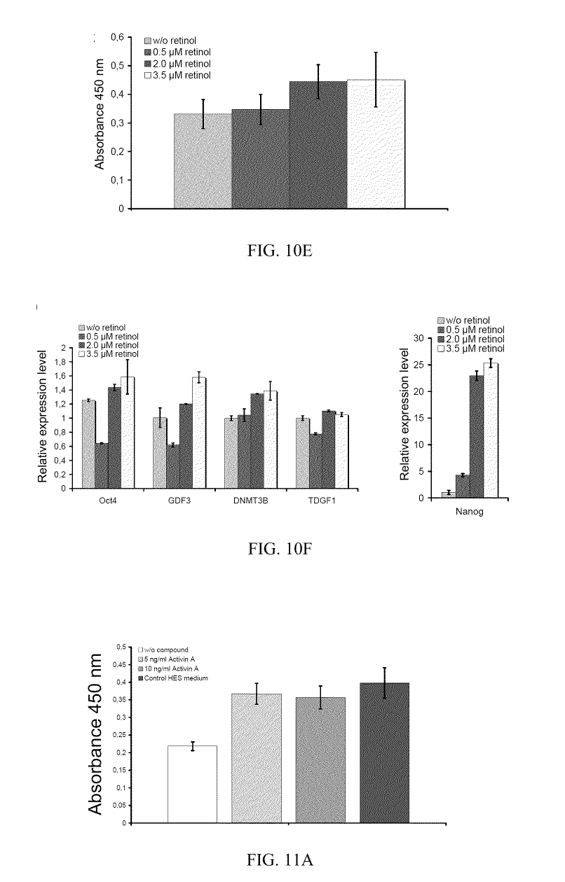

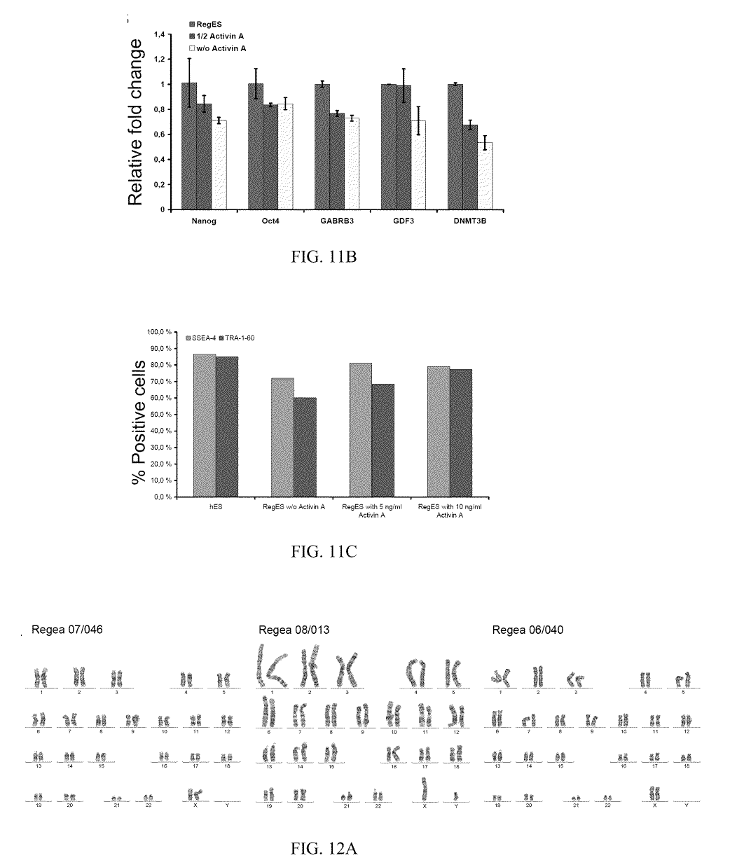

Retinol was selected to be further evaluated in the maintenance of undifferentiated hESCs. Initial studies showed that retinol at a concentration of 0.1-0.5 μM was not effective and no improvement in the morphology or in the number of undifferentiated colonies was seen. Further evaluation, however, showed that retinol at a concentration of 2.0 μM or above improved the proliferation of hESCs as well as induced the expression of hESC specific markers (FIG. 10). In the presence of 2.0 μM retinol, the growth of the colonies started earlier and already at day 3 the size of the colonies was bigger (FIG. 10A-10B). Proliferation assay demonstrated that hESCs cultured in the presence of 2.0 μM or 3.5 μM retinol had almost two-fold proliferation rate when compared to hESCs cultured without retinol or in the presence of 0.5 μM retinol (FIG. 10E). Immunocytochemical staining of hESCs cultured in the presence of retinol showed expression of stem cell markers Nanog and TRA-1-81 (FIG. 10C-10D). Furthermore, retinol increased the expression of pluripotency supporting genes, especially Nanog, which relative expression level was over twentyfold in the presence of 2.0 μM and 3.5 μM retinol (FIG. 10F).

### EXAMPLE 8

### Activin A Further Enhances the Performance of the Present Culture

Proliferation assay demonstrated that hESCs cultured in the presence of 5 or 10 ng/ml Activin A in the present culture medium had almost two-fold proliferation rate when compared to hESCs cultured without Activin A and the proliferation rate was comparable to hESCs cultured in the control hES medium (FIG. 11A). Fluorescence-activated cell sorting (FACS) and quantitative reverse transcription PCR (qRT-PCR) analysis demonstrated that Activin A increased the expression of pluripotency supporting markers at both transcriptional and translational level (FIGS. 11B-11C).

### EXAMPLE 9

### Derivation, Long-term Culture and Characterization of hESCs in the Present Culture Medium

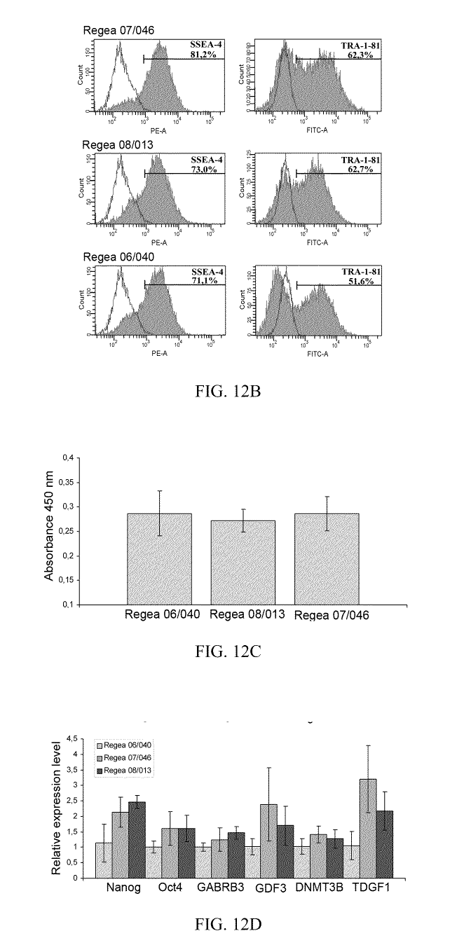

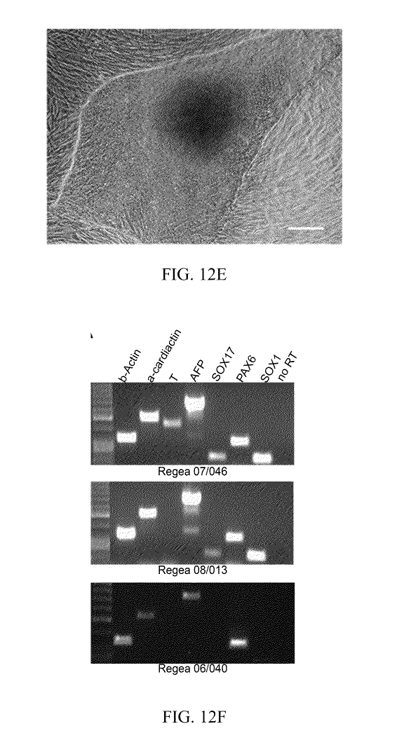

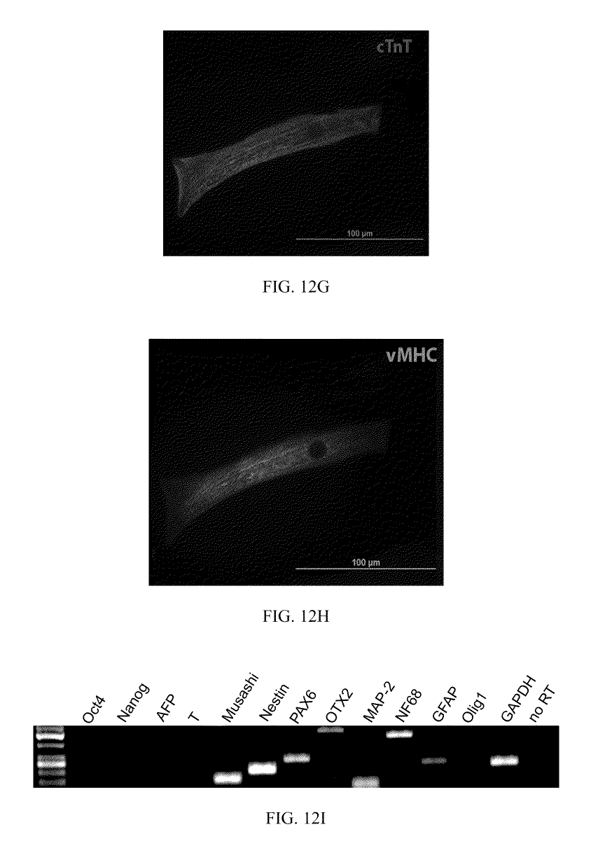

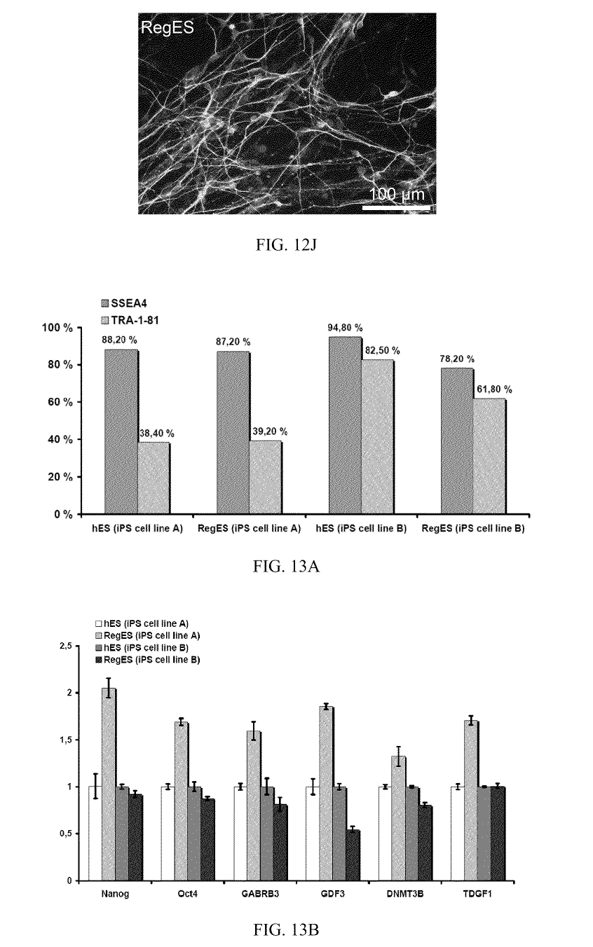

Using the present culture medium comprising retinol and Activin A, new clinical-grade hESC lines (07/046 and 08/013) have been successfully derived from surplus bad quality human embryo donated for stem cell research. Human ESC lines have been continuously cultured for over 80 passages. These cell lines have been karyotyped regularly and exhibit a normal diploid karyotype (FIG. 12A). Fluorescence-activated cell sorting (FACS) and quantitative reverse transcription PCR (qRT-PCR) analysis demonstrated that these cell lines express stem cell markers at levels comparable to the hESC line Regea 06/040 derived and cultured using hES medium (FIG. 12B, D). Cell proliferation analysis showed that the cell proliferation rates of Regea 07/046 and Regea 08/013 cell lines were comparable to that of Regea 06/040 (FIG. 12C). The present culture medium may also be used for freezing and thawing of the hESCs (FIG. 12E).

To confirm that the new cell lines maintain their pluripotency in vitro, we performed an embryoid body (EB) assay. The EB-derived cells from the cell lines Regea 07/046 and 08/013 expressed markers from the three different embryonic lineages; endoderm, ectoderm, and mesoderm (FIG. 12F). We also tested whether hESCs derived and cultured for long-term in xeno-free conditions can differentiate to cardiomyocytes and neural cell lineages. Spontaneously beating areas were observed after 12-16 days after the initiation of the cardiac differentiation. Dissociated, spontaneously beating cells had striated patterning and were positively stained with cardiac troponin T and ventricular myosin heavy chain markers (FIG. 12G-12H). To generate neuronal cells, hESC colonies cultured in the culture medium according to the present invention and hES media were dissected into small clusters and cultured in suspension for up to 20 weeks. The differentiated cells expressed neural precursor markers, neuronal markers and astrocytic marker in RT-PCR (FIG. 12I). Immunocytochemical staining verified the neuronal and glial fate of the cells (FIG. 12J). These results indicated that hESC lines derived and cultured in the present xeno-free culture medium maintain their pluripotency and furthermore cardiomyocytes and neuronal cells can be generated from these cell lines.

Similar clinical-grade hESCs have been successfully derived also with the present culture medium according to other embodiments described herein.

### EXAMPLE 10

### Culture and Characterization of iPS Cells in the Present Culture Medium

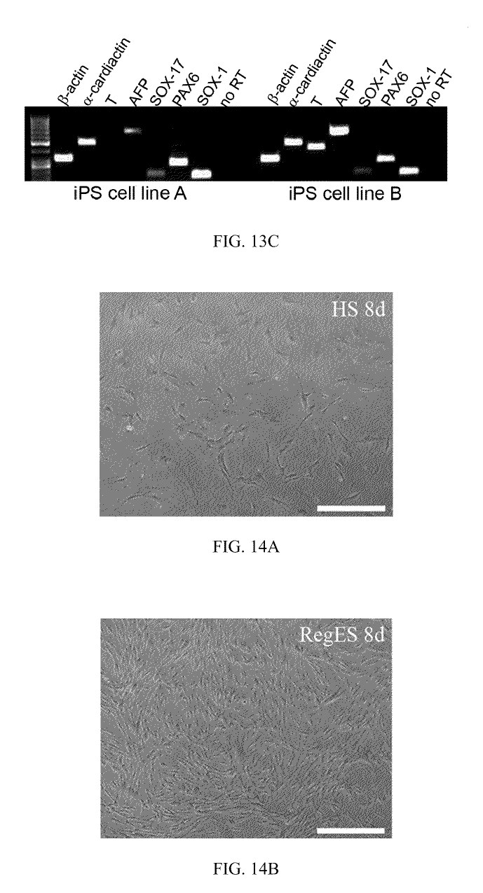

To further demonstrate the performance of the present culture medium comprising retinol and Activin A developed for human pluripotent cells, we cultured two human induced pluripotent stem cell (iPS cell) lines on human feeder cells in these conditions. The morphology and stem cell marker expression of the cells were similar as compared to the cells cultured in hES medium (FIG. 13A-13B). In addition, analysis of EBs demonstrated that iPS cells cultured in the present culture medium maintained their ability to differentiate to all three germ layers (FIG. 13C).

Similar results have been obtained with the present culture medium according to other embodiments of the present invention.

### EXAMPLE 11

### Culture and Characterization of ASCs in the Present Culture Medium

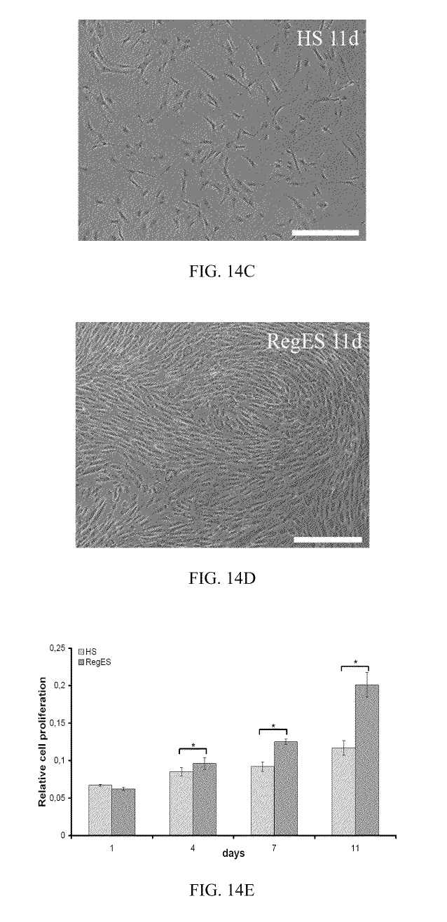

ASCs isolated from adipose tissue samples were used to assess the performance of the present culture medium comprising retinol and Activin A for the culture of mesenchymal stem cells. To determine the proliferation rate of ASCs grown in the present culture medium and human serum containing medium (HS medium) the WST-1 proliferation analysis was performed at several time points (1, 4, 7 and 11 days). Seven ASC lines were used for the analysis in both conditions. Concurrently, cell morphology was observed by light microscopic examination to confirm the proliferation assay results (FIG. 14A-14D). The proliferation analysis showed already at day 4 that cultures with the present culture medium exhibited a higher proliferation rates of ASCs as compared to HS medium (FIG. 14E). Subsequently, ASCs continued to proliferate at a higher rate in the present culture medium compared to HS medium at day 7 and 11. Significant differences in the proliferation rates were observed between the present culture medium and HS medium at day 4 (p=0.035), day 7 (p=0.022) and day 11 (p=0.018) (FIG. 14E).

Flow cytometric characterization was performed to compare surface marker expression characteristics of ASCs expanded in the present culture medium and HS medium (Table 8). Four cell lines were analyzed for every culture condition. While both culture conditions maintained the characteristic surface marker expression profile of ASCs, statistical analysis revealed significant differences in the expression of sialomucin-like adhesion molecule CD34 (p=0.043), leucocyte common antigen CD45 (p=0.017), adhesion molecule CD105 (p=0.020) and MHC Class I isotype HLA-ABC (p=0.021) of ASCs cultured in HS medium and in the culture medium according to the present invention.

TABLE 8
Surface marker expression characteristics of ASCs cultured
in HS medium and in the present culture medium

| | | HS | RegES |
|---|---|---|---|
| Surface | | medium | medium |
| Protein | Antigen | (n = 4) | (n = 4) |
| CD34 * | Sialomucin-like adhesion | 3.5 ± 1.7 | 1.2 ± 0.7 |
| | molecule | | |
| CD45 * | Leukocyte common antigen | 0.4 ± 0.0 | 2.4 ± 1.2 |
| CD90 | Thy-1, T-cell surface | 93.1 ± 11.2 | 99.8 ± 0.1 |
| | glycoprotein | | |
| CD105 * | SH-2, endoglin | 52.0 ± 8.3 | 75.7 ± 6.6 |
| HLA-ABC * | Major histocompatibility | 0.6 ± 0.4 | 10.0 ± 11.4 |
| | class I antigen | | |
| HLA-DR | Major histocompatibility | 0.8 ± 0.6 | 0.4 ± 0.1 |
| | class II antigen | | |

Cell lines 5/08, 19/08, 24/08 and 25/08 cultured in HS medium were at passage 2-3 and cell lines 9/08, 11/08, 25/08 and 31/08 cultured in the culture medium according to the present invention were at passage 3-4. Data are presented as mean±standard deviation from the number of donors/samples indicated in parentheses. *p<0.05.

It will be obvious to a person skilled in the art that, as the technology advances, the inventive concept can be implemented in various ways. The invention and its embodiments are not limited to the examples described above but may vary within the scope of the claims.

All references cited are included herein by reference.

1. A xeno-free serum replacement comprising at least one fatty acid selected from a group consisting of conjugated linoleic acid and eicosapentaenoic acid.

2. The serum replacement according to claim 1, further comprising Activin A.

3. The serum replacement according to claim 1, further comprising retinol.

4. The serum replacement according to claim 1 further comprising stearic acid.

5. The serum replacement according to claim 1, wherein, the concentration of conjugated linoleic acid (CLA) is such that a final culture medium, which is a basal medium supplemented with said serum replacement, comprises from about 0.5 mg/l to about 5 mg/l CLA, and the concentration of eicosapentaenoic acid (EPA) is such that the final culture medium comprises from about 1 mg/l to about 10 mg/l EPA.

6. The serum replacement according to claim 2, wherein the concentration of Activin A is such that a final culture medium, which is a basal medium supplemented with said serum replacement, comprises from about 0.001 mg/l to about 0.02 mg/l Activin A.

7. The serum replacement according to claim 3, wherein the concentration of retinol is such that a final culture medium, which is a basal medium supplemented with said serum replacement, comprises from about 0.25 mg/l to about 0.5 mg/l retinol.

8. The serum replacement according to claim 4, wherein the concentration of stearic acid is such that a final culture medium, which is a basal medium supplemented with said serum replacement, comprises from about 0.5 mg/l to about 5 mg/l stearic acid.

9. A xeno-free cell culture medium comprising a basal medium and the serum replacement according to claim 1.

10. A method for initiating a new stem cell line in vitro, comprising
a) providing isolated cells of desired origin,
b) contacting said cells with the xeno-free medium according to claim 9, and
c) cultivating said cells under conditions suitable for stem cell culture.

11. The method according to claim 10, wherein said isolated cells are of embryonic, adult somatic, or mesenchymal origin.

12. A method for culturing stem cells, comprising
a) contacting said stem cells with the xeno-free medium according to claim 9, and
b) cultivating said cells under conditions suitable for stem cell culture.

13. The method according to claim 11, wherein said cultivation is performed on a feeder cell layer.

14. A method for differentiating stem cells, comprising
a) contacting said stem cells with the xeno-free medium according to claim 9 supplemented with a differentiating agent, and
b) cultivating said cells under conditions suitable for differentiation of stem cells.

15. The serum replacement according to claim 2, further comprising retinol.

16. The serum replacement according to claim 15, wherein the concentration of retinol is such that a final culture medium, which is a basal medium supplemented with said serum replacement, comprises from about 0.25 mg/l to about 0.5 mg/l retinol.

17. The method according to claim 12, wherein said cultivation is performed on a feeder cell layer.
